(19) United States
(12) Patent Application Publication
(10) Pub. No.: US 2009/0181929 A1
(43) Pub. Date: Jul. 16, 2009

(54) ORGANIC COMPOUNDS

(76) Inventors: Kazuhide Konishi, Ibaraki (JP); Yuki Iwaki, Ibaraki (JP)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/300,385

(22) PCT Filed: May 10, 2007
(86) PCT No.: PCT/JP2007/060086

§ 371 Date: Nov. 11, 2008

Related U.S. Application Data
(60) Provisional application No. 60/799,445, filed on May 11, 2006.

(51) Int. Cl. A61K 31/4439; C07D 409/14; C07D 401/12; C07D 401/14; A61K 31/4545; A61K 31/444; A61K 31/695; A61P 3/00; A61P 9/00

(52) U.S. Cl. 514/63; 546/268.4; 546/256; 546/194; 546/278.4; 514/318; 514/340; 514/333; 514/343; 514/210.2

(57) ABSTRACT

The present invention provides a compound of formula (I):

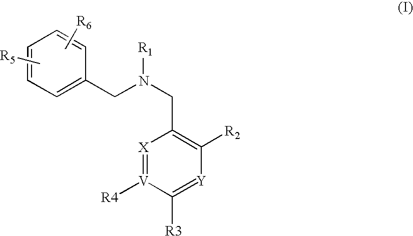

said compound is an inhibitor of CETP, and thus can be employed for the treatment of a disorder or disease mediated by CETP or responsive to the inhibition of CETP.

The present invention provide a novel compound of formula (I):

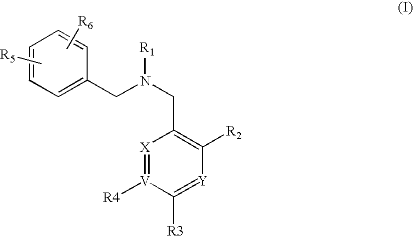

wherein

X and Y are independently CH or N;

V is C or N, provided that when V is N, R4 is hydrogen;

R₁ is heteroaryl, heterocyclyl, aryl, alkoxycarbonyl, alkanoyl, or alkyl, each is optionally substituted with one to three substituents selected from alkyl, hydroxy, halogen, nitro, carboxy, thiol, cyano, HSO₃—, cycloalkyl, alkenyl, alkoxy, cycloalkoxy, alkenyloxy, alkoxycarbonyl, carbamimidoyl, alkyl-S—, alkyl-SO—, alkyl-SO₂—, amino, H₂N—SO₂—, alkanoyl, heterocyclyl;

R₂ is hydrogen, alkyl, halogen, cycloalkyl, cycloalkyl-alkyl—, aryl, alkoxy, or (R₇)(R₈)N—;

wherein R₇ and R₈ are independently alkyl, cycloalkyl, alkanoyl, cycloalkyl-C(O)—, or

R₉-alkyl-, each of which is optionally substituted by one to three substituents selected from alkyl, alkanoyl, hydroxy, alkoxy, or halogen;

wherein R₉ is aryl, cycloalkyl, heterocyclyl, R₁₀—C(O);

wherein R₁₀ is hydrogen, hydroxy, alkyl, heterocyclyl, (Rₐ)(R_{b})N— or cycloalkyl;

wherein Rₐ and R_{b} is alkyl, cycloalkyl, alkanoyl, cycloalkyl-C(O)—,

R₇ and R₈ taken together with the nitrogen to which they are attached optionally form a 3 to 8 membered ring; or

R₂ is heterocyclyl that is optionally substituted by one to three substituents selected from alkyl, hydroxy, aryl, aryl-alkyl-, cycloalkyl, heteroaryl, heterocyclyl, halogen, carboxy, amide, SO₂NH, alkyl-SO₂—NH—, alkyl-NH—SO₂—, or R₁₀—C(O)—, wherein R₁₀ is hydrogen, hydroxy, alkyl, heterocyclyl, (R₇)(R₈)N— or cycloalkyl;

R₃ is aryl or heteroaryl, each is optionally substituted by one to two substituents selected from halogen, alkyl, alkoxy, or alkyl-SO₂;

R₄ is substituted aryl or heteroaryl, each is substituted by one to two substituents selected from halogen, alkyl, alkoxy, or alkyl-SO₂; or

R₃ and R₄ are independently hydrogen, alkyl, alkoxy, halogen, heterocyclyl, alkyl-S—, alkyl-SO₂, aryloxy, cyano, nitro, HO—C(O)—, or hydroxy; or

R₃ and R₄ are independently (R₁₁)(R₁₂)N—C(O)—, (R₁₃)(R₁₄)N—, wherein R₁₁ and R₁₂ are independently hydrogen, alkyl, aryl, heteroary, or aryl-alkyl—; R₁₃ and R₁₄ are independently hydrogen, alkyl, alkyl-C(O)—, or alkyl-SO₂—;

R₁₃ and R₁₄ taken together with the nitrogen to which they are attached optionally form a 3 to 8 membered ring;

R₅ and R₆ are independently hydrogen, alkyl, haloalkyl, halogen, cyano, nitro, hydroxy, or alkoxy; or

R₆ is aryl or heteroaryl; or

a pharmaceutically acceptable salt thereof; or an optical isomer thereof; or a mixture of optical isomers.

In one embodiment, the present invention provides the compound of formula (I), wherein

X and Y are independently CH or N;

V is C or N, provided that when V is N, R₄ is hydrogen;

R₁ is (5-9)-membered heteroaryl, (5-9)-membered heterocyclyl, (C₆-C₁₀) aryl, or (C₁-C₇) alkyl, each is optionally substituted with one substituent selected from (C₁₋C₇) alkyl, hydroxy, halogen, nitro, carboxy, thiol, cyano, HSO₃—, (C₃-C₇) cycloalkyl, <C₁-C₇) alkenyl, (C₁-C₇) alkoxy, (C₃-C₇) cycloalkoxy, (C₁-C₇) alkenyloxy, (C₁-C₇) alkoxycarbonyl, carbamimidoyl, (C₁-C₇) alkyl-S—, (C₁-C₇) alkyl-SO—, (C₁-C₇) alkyl-SO₂—, amino, H₂N—SO₂—, (C₁-C₇) alkanoyl, (5-9)-membered heterocyclyl;

R₂ is hydrogen, (C₁-C₇) alkyl, halogen, (C₃-C₇) cycloalkyl, (C₃-C₇) cycloalkyl-(C₁-C₇) alkyl, (C₆₋C₁₀) aryl, (C₁-C₇) alkoxy, (5-9)-membered heterocyclyl, or (R₇)(R₈)N—, wherein R₇ and R₈ are independently (C₁-C₇) alkyl, hydroxy, halogen, (C₁-C₇) alkyl-C(O)—, (C₃-C₇) cycloalkyl-C(O)—, or R₉—(C₁-C₇) alkyl-, wherein R₉ is (C₃-C₇) cycloalkyl, (C₆-C₁₀) aryl, (5-9)-membered heterocyclyl, or R₁₀—C(O), wherein R₁₀ is (C₃-C₇) cycloalkyl, (C₁-C₇) alkyl, (5-9)-membered heterocyclyl, (Rₐ)(R_{b})N—, hydroxy, or hydrogen;

wherein Rₐ and R_{b} is (C₁-C₇) alkyl, (C₃-C₇) cycloalkyl, (C₁-C₇) alkanoyl, (C₃-C₇) cycloalkyl-C(O)—,

R₇ and R₈ taken together with the nitrogen to which they are attached optionally form a 3 to 8 membered ring; or

R₂ is (5-9)-membered heterocyclyl that is optionally substituted by one to two substituents selected from (C₁-C₇) alkyl, hydroxy, (C₆-C₁₀) aryl, (C₆-C₁₀) aryl-(C₁-C₇) alkyl-, (C₃-C₇) cycloalkyl, (5-9)-membered heteroaryl, carboxy, amide, SO₂—NH—, (C₁-C₇) alkyl-SO₂—NH—, (C₁-C₇) alkyl-NH—SO₂—, halogen, or R₁₀—C(O), wherein R₁₀ is (C₃-C₇) cycloalkyl, (C₁-C₇) alkyl, hydroxy, or hydrogen;

R₃ is (C₆-C₁₀) aryl or (5-9)-membered heteroaryl, each is optionally substituted by one to two substituents selected from halogen, (C₁-C₇) alkyl, (C₁-C₇) alkoxy, or (C₁-C₇) alkyl-SO₂—;

R₄ is substituted (C₆-C₁₀) aryl or (5-9)-membered heteroaryl, each is optionally substituted by one to two substituents selected from halogen, (C₁-C₇) alkyl, (C₁-C₇) alkoxy, or (C₁-C₇) alkyl-SO₂; or

R₃ and R₄ are independently hydrogen, (C₁-C₇) alkyl, (C₁-C₇) alkoxy, halogen, (5-9)-membered heterocyclyl, (C₁-C₇) alkyl-S—, (C₁-C₇) alkyl-SO₂—, (C₆-C₁₀) aryloxy, cyano, nitro, HO—C(O)—, or hydroxy; or

R₃ and R₄ are independently (R₁₀)(R₁₁)N—C(O)—, (R₁₂)(R₁₃)N, wherein R₁₀ and R₁₁ are independently hydrogen or (C₁-C₇) alkyl, (C₆-C₁₀) aryl, (C₆-C₁₀) aryl-(C₁-C₇) alkyl-; R₁₂ and R₁₃ are independently hydrogen, (C₁-C₇) alkyl, (C₁-C₇) alkyl-C(O)—, or (C₁-C₇) alkyl-SO₂—;

R₁₂ and R₁₃ taken together with the nitrogen to which they are attached optionally form a 3 to 8 membered ring;

R₅ and R₆ are independently hydrogen, (C₁-C₇) alkyl, (C₁-C₇) haloalkyl, halogen, cyano, nitro, hydroxy, or (C₁-C₇) alkoxy; or

R₆ is (C₆-C₁₀) aryl or (5-9)-membered heteroaryl; or

a pharmaceutical acceptable salt thereof; or an optical isomer thereof; or a mixture of optical isomers.

For purposes of interpreting this specification, the following definitions will apply and whenever appropriate, terms used in the singular will also include the plural and vice versa.

As used herein, the term “alkyl” refers to a fully saturated branched or unbranched hydrocarbon moiety. Preferably the alkyl comprises 1 to 20 carbon atoms, more preferably 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. When an alkyl group includes one or more unsaturated bonds, it can be referred to as an alkenyl (double bond) or an alkynyl (triple bond) group.

The term “aryl” refers to monocyclic or bicyclic aromatic hydrocarbon groups having 6-20 carbon atoms in the ring portion. Preferably, the aryl is a (C₆-C₁₀) aryl. Non-limiting examples include phenyl, biphenyl, naphthyl or tetrahydronaphthyl, each of which may optionally be substituted by 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S—, nitro, cyano, carboxy, alkyl-O—C(O)—, carbamoyl, alkyl-S(O)—, sulfonyl, sulfonamide, heterocyclyl and the like, wherein R is independently hydrogen, alkyl, aryl, heteroaryl, aryl-alkyl-, heteroaryl-alkyl- and the like.

Furthermore, the term “aryl” as used herein, refers to an aromatic substituent which can be a single aromatic ring, or multiple aromatic rings that are fused together, linked covalently, or linked to a common group such as a methylene or ethylene moiety. The common linking group also can be a carbonyl as in benzophenone or oxygen as in diphenylether or nitrogen as in diphenylamine.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like. Preferably, alkoxy groups have about 1-7, more preferably about 1-4 carbons.

As used herein, the term “acyl” refers to a group R—C(O)— of from 1 to 10 carbon atoms of a straight, branched, or cyclic configuration or a combination thereof, attached to the parent structure through carbonyl functionality. Such group can be saturated or unsaturated, and aliphatic or aromatic. Preferably, R in the acyl residue is alkyl, or alkoxy, or aryl, or heteroaryl. Also preferably, one or more carbons in the acyl residue may be replaced by nitrogen, oxygen or sulfur as long as the point of attachment to the parent remains at the carbonyl. Examples include but are not limited to, acetyl, benzoyl, propionyl, isobutyryl, t-butoxycarbonyl, benzyloxycarbonyl and the like. Lower acyl refers to acyl containing one to four carbons.

As used herein, the term “acylamino” refers to acyl-NH—, wherein “acyl” is defined herein.

As used herein, the term “carbamoyl” refers to H₂NC(O)—, alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aryl-alkyl-NHC(O)—, alkyl(aryl-alkyl)-NC(O)— and the like.

As used herein, the term “sulfonyl” refers to R—SO₂—, wherein R is hydrogen, alkyl, aryl, hereoaryl, aryl-alkyl, heteroaryl-alkyl, aryl-O—, heteroaryl-O—, alkoxy, aryloxy, cycloalkyl, or heterocyclyl.

As used herein, the term “sulfonamide” refers to alkyl-S(O)₂—NH—, aryl-S(O)₂—NH—, aryl-alkyl-S(O)₂—NH—, heteroaryl-S(O)₂—NH—, heteroaryl-alkyl-S(O)₂—NH—, alkyl-S(O)₂—N(alkyl)-, aryl-S(O)₂—N(alkyl)-, aryl-alkyl-S(O)₂—N(alkyl)-, heteroaryl-S(O)₂—N(alkyl)-, heteroaryl-alkyl-S(O)₂—N(alkyl)- and the like.

As used herein, the term “alkoxycarbonyl” refers to alkoxy-C(O)—, wherein alkoxy is defined herein.

As used herein, the term “alkanoyl” refers to alkyl-C(O)—, wherein alkyl is defined herein.

As used herein, the term “alkenyl” refers to a straight or branched hydrocarbon group having 2 to 20 carbon atoms and that contains at least one double bonds. The alkenyl groups preferably have about 2 to 8 carbon atoms.

As used herein, the term “alkenyloxy” refers to alkenyl-O—, wherein alkenyl is defined herein.

As used herein, the term “cycloalkoxy” refers to cycloalkoxy-O—, wherein cycloalkyl is defined herein.

As used herein, the term “carbamimidoyl” refers to H₂NC(NH)—.

As used herein, the term “heterocyclyl” or “heterocyclo” refers to an optionally substituted, fully saturated or unsaturated, aromatic or nonaromatic cyclic group, e.g., which is a 4- to 7-membered monocyclic, 7- to 12-membered bicyclic or 10- to 15-membered tricyclic ring system, which has at least one heteroatom in at least one carbon atom-containing ring. Each ring of the heterocyclic group containing a heteroatom may have 1, 2or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, triazolyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, 1,1,4-trioxo-1,2,5-thiadiazolidin-2-yl and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]-pyridinyl] or furo[2,3-b]pyridinyl), dihydroisoindolyl, 1,3-dioxo-1,3-dihydroisoindol-2-yl, dihydroquinazolinyl (such as 3,4-dihydro-4-oxo-quinazolinyl), phthalazinyl and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

The term “heterocyclyl” further refers to heterocyclic groups as defined herein substituted with 1, 2 or 3 substituents selected from the groups consisting of the following:

(a) alkyl;

(b) hydroxy (or protected hydroxy);

(c) halo;

(d) oxo, i.e., ═O;

(e) amino, alkylamino or dialkylamino;

(f) alkoxy;

(g) cycloalkyl;

(h) carboxy;

(i) heterocyclooxy, wherein heterocyclooxy denotes a heterocyclic group bonded through an oxygen bridge;

(j) alkyl-O—C(O)—;

(k) mercapto;

(l) nitro;

(m) cyano;

(n) sulfamoyl or sulfonamido;

(o) aryl;

(p) alkyl-C(O)—O—;

(q) aryl-C(O)—O—;

(r) aryl-S—;

(s) aryloxy;

(t) alkyl-S—;

(u) formyl, i.e., HC(O)—;

(v) carbamoyl;

(w) aryl-alkyl-; and

(x) aryl substituted with alkyl, cycloalkyl, alkoxy, hydroxy, amino, alkyl-C(O)—NH—, alkylamino, dialkylamino or halogen.

As used herein, the term “cycloalkyl” refers to optionally substituted saturated or unsaturated monocyclic, bicyclic or tricyclic hydrocarbon groups of 3-12 carbon atoms, each of which may be substituted by one or more substituents, such as alkyl, halo, oxo, hydroxy, alkoxy, alkyl-C(O)—, acylamino, carbamoyl, alkyl-NH—, (alkyl)₂N—, thiol, alkylthio, nitro, cyano, carboxy, alkyl-O—C(O)—, sulfonyl, sulfonamido, sulfamoyl, heterocyclyl and the like. Exemplary monocyclic hydrocarbon groups include, but are not limited to, cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like. Exemplary tricyclic hydrocarbon groups include adamantyl and the like.

As used herein, the term “sulfamoyl” refers to H₂NS(O)₂—, alkyl-NHS(O)₂—, (alkyl)₂NS(O)₂—, aryl-NHS(O)₂—, alkyl(aryl)-NS(O)₂—, (aryl)₂NS(O)₂—, heteroaryl-NHS(O)₂—, aralkyl-NHS(O)₂—, heteroaralkyl-NHS(O)₂— and the like.

As used herein, the term “aryloxy” refers to both an —O-aryl and an —O— heteroaryl group, wherein aryl and heteroaryl are defined herein.

As used herein, the term “heteroaryl” refers to a 5-14 membered monocyclic- or bicyclic- or fused polycyclic-ring system, having 1 to 8 heteroatoms selected from N, O or S. Preferably, the heteroaryl is a 5-10 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl.

The term “heteroaryl” also refers to a group in which a heteroaromatic ring is fused to one or more aryl, cycloaliphatic, or heterocyclyl rings, where the radical or point of attachment is on the heteroaromatic ring. Nonlimiting examples include but are not limited to 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinoliyl, 1-, 3-, 4-, 5-, 6-, 7-, or 8-isoquinoliyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4aH carbazolyl, 1-, 2-, 3-, 4-, 5-, 6-, 7-, or 8-carbzaolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or l-, 3-, 4-, 5-, 6-, 7-, 8-, 9-, or 10-benzisoqinolinyl, 2-, 3-, 4-, or thieno[2,3-b]furanyl, 2-, 3-, 5-, 6-, 7-, 8-, 9-, 10-, or 11-7H-pyrazino[2,3-c]carbazolyl, 2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6-, 7-, 8-, or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroary groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl.

A heteroaryl group may be mono-, bi-, tri-, or polycyclic, preferably mono-, bi-, or tricyclic, more preferably mono- or bicyclic.

As used herein, the term “halogen” or “halo” refers to fluoro, chloro, bromo, and iodo.

As used herein, the term “haloalkyl” refers to an alkyl as defined herein, that is substituted by one or more halo groups as defined herein. Preferably the haloalkyl can be monohaloalkyl, dihaloalkyl or polyhaloalkyl including perhaloalkyl. A monohaloalkyl can have one iodo, bromo, chloro or fluoro within the alkyl group. Dihaloalky and polyhaloalkyl groups can have two or more of the same halo atoms or a combination of different halo groups within the alkyl. Preferably, the polyhaloalkyl contains up to 12, 10, or 8, or 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

As used herein, the term “isomers” refers to different compounds that have the same molecular formula. Also as used herein, the term “an optical isomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “pharmaceutically acceptable salts” refers to salts that retain the biological effectiveness and properties of the compounds of this invention and, which are not biologically or otherwise undesirable. Non-limiting examples of the salts include non-toxic, inorganic and organic base or acid addition salts of compounds of the present invention. In many cases, the compounds of the present invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in Remington's Pharmaceutical Sciences, 20th ed., Mack Publishing Company, Easton, Pa., (1985), which is herein incorporated by reference.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The term “therapeutically effective amount” of a compound of the present invention refers to an amount of the compound of the present invention that will elicit the biological or medical response of a subject, or ameliorate symptoms, slow or delay disease progression, or prevent a disease, etc. In a preferred embodiment, the “effective amount” refers to the amount that inhibits or reduces expression or activity of CETP.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “a disorder” or “a disease” refers to any derangement or abnormality of function; a morbid physical or mental state. See Dorland's Illustrated Medical Dictionary, (W.B. Saunders Co. 27th ed. 1988).

As used herein, the term “inhibition” or “inhibiting” refers to the reduction or suppression of a given condition, symptom, or disorder, or disease, or a significant decrease in the baseline activity of a biological activity or process. Preferably, the condition or symptom or disorder or disease is mediated by CETP activity or responsive to the inhibition of CETP.

As used herein, the term “treating” or “treatment” of any disease or disorder refers in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

As used herein, the term “a,” “an,” “the” and similar terms used in the context of the present invention (especially in the context of the claims) are to be construed to cover both the singular and plural unless otherwise indicated herein or clearly contradicted by the context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Any asymmetric carbon atom on the compounds of the present invention can be present in the (R)-, (S)- or (R,S)-configuration, preferably in the (R)- or (S)-configuration. Substituents at atoms with unsaturated bonds may, if possible, be present in cis- (Z)- or trans-(E)-form. Therefore, the compounds of the present invention can be in the form of one of the possible isomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, the imidazolyl moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Finally, compounds of the present invention are either obtained in the free form, as a salt thereof, or as prodrug derivatives thereof.

When a basic group is present in the compounds of the present invention, the compounds can be converted into acid addition salts thereof, in particular, acid addition salts with the imidazolyl moiety of the structure, preferably pharmaceutically acceptable salts thereof. These are formed, with inorganic acids or organic acids. Suitable inorganic acids include but are not limited to, hydrochloric acid, sulfuric acid, a phosphoric or hydrohalic acid. Suitable organic acids include but are not limited to, carboxylic acids, such as (C₁-C₄)alkanecarboxylic acids which, for example, are unsubstituted or substituted by halogen, e.g., acetic acid, such as saturated or unsaturated dicarboxylic acids, e.g., oxalic, succinic, maleic or fumaric acid, such as hydroxycarboxylic acids, e.g., glycolic, lactic, malic, tartaric or citric acid, such as amino acids, e.g., aspartic or glutamic acid, organic sulfonic acids, such as (C₁-C₄)alkylsulfonic acids, e.g., methanesulfonic acid; or arylsulfonic acids which are unsubstituted or substituted, e.g., by halogen. Preferred are salts formed with hydrochloric acid, methanesulfonic acid and maleic acid.

When an acidic group is present in the compounds of the present invention, the compounds can be converted into salts with pharmaceutically acceptable bases. Such salts include alkali metal salts, like sodium, lithium and potassium salts; alkaline earth metal salts, like calcium and magnesium salts; ammonium salts with organic bases, e.g., trimethylamine salts, diethylamine salts, tris(hydroxymethyl)methylamine salts, dicyclohexylamine salts and N-methyl-D-glucamine salts; salts with amino acids like arginine, lysine and the like. Salts may be formed using conventional methods, advantageously in the presence of an ethereal or alcoholic solvent, such as a lower alkanol. From the solutions of the latter, the salts may be precipitated with ethers, e.g., diethyl ether. Resulting salts may be converted into the free compounds by treatment with acids. These or other salts can also be used for purification of the compounds obtained.

When both a basic group and an acid group are present in the same molecule, the compounds of the present invention can also form internal salts.

The present invention also provides pro-drugs of the compounds of the present invention that converts in vivo to the compounds of the present invention. A pro-drug is an active or inactive compound that is modified chemically through in vivo physiological action; such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds are inactive or have low activity compared to the corresponding active drug compound, that contains one or more protective groups and are converted to an active form by metabolism or solvolysis. Both the active drug form and any released metabolic products should have acceptably low toxicity. Typically, the formation of active drug compound involves a metabolic process or reaction that is one of the follow types:

1. Oxidative reactions, such as oxidation of alcohol, carbonyl, and acid functions, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-delakylation, oxidative O- and S-delakylation, oxidative deamination, as well as other oxidative reactions.

2. Reductive reactions, such as reduction of carbonyl groups, reduction of alcoholic groups and carbon-carbon double bonds, reduction of nitrogen-containing functions groups, and other reduction reactions.

3. Reactions without change in the state of oxidation, such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. See, Cheng et al., US20040077595, application Ser. No. 10/656,838, incorporated herein by reference. Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxy groups with lipophilic carboxylic acids, or of carboxylic acid groups with alcohols, e.g., aliphatic alcohols. Wermuth, The Practice of Medicinal Chemistry, Ch. 31-32, Ed. Werriuth, Academic Press, San Diego, Calif., 2001.

Exemplary prodrugs are, e.g., esters of free carboxylic acids and S-acyl and O-acyl derivatives of thiols, alcohols or phenols, wherein acyl has a meaning as defined herein. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

In view of the close relationship between the compounds, the compounds in the form of their salts and the pro-drugs, any reference to the compounds of the present invention is to be understood as referring also to the corresponding pro-drugs of the compounds of the present invention, as appropriate and expedient.

Furthermore, the compounds of the present invention, including their salts, can also be obtained in the form of their hydrates, or include other solvents used for their crystallization.

The compounds of the present invention have valuable pharmacological properties.

The compounds of the present invention are useful as inhibitors for cholesteryl ester transfer protein (CETP). CETP is a 74 KD glycopeptide, it is secreted by the liver and is a key player in facilitating the transfer of lipids between the various lipoproteins in plasma. The primary function of CETP is to redistribute cholesteryl esters (CE) and triglycerides between lipoproteins. See Assmann, G et al., “HDL cholesterol and protective factors in atherosclerosis,” Circulation, 109:1118-1114 (2004). Because most triglycerides in plasma originate in VLDLs and most CEs are formed in HDL particles in the reaction catalyzed by lecithin:cholesterol acyltransferase, activity of CETP results in a net mass transfer of triglycerides from VLDLs to LDLs and HDLs and a net mass transfer of CEs from HDLs to VLDLs and LDLs. Thus, CETP potentially decreases HDL-C levels, increases LDL-cholesteryl (LDL-C) levels and reduces HDL and LDL particles size, and inhibition of CETP could be a therapeutic strategy for raising HDL-cholesteryl (HDL-C), have a favorable impact on the lipoprotein profile, and reduce the risk of cardiovascular diseases. Accordingly, the compounds of the present invention as CETP inhibitors are useful for the delay of progression and/or treatment of a disorder or disease that is mediated by CETP or responsive to inhibition of CETP. Disorders, conditions and diseases that can be treated with the compounds of the present invention include but are not limited to, hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity, infection or egg embryonation of schistosoma, or endotoxemia etc.

Additionally, the present invention provides:

a compound of the present invention as described herein above for use as a medicament;

the use of a compound of the present invention as described herein above for the preparation of a pharmaceutical composition for the delay of progression and/or treatment of a disorder or disease mediated by CETP, or responsive to inhibition of CETP.

the use of a compound of the present invention as described herein above for the preparation of a pharmaceutical composition for the delay of progression and/or treatment of a disorder or disease selected from hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity or endotoxemia etc.

The compounds of formula (I) can be prepared by the procedures described in the following sections.

Generally, the compounds of formula (I) can be prepared according to the following general procedures and schemes.

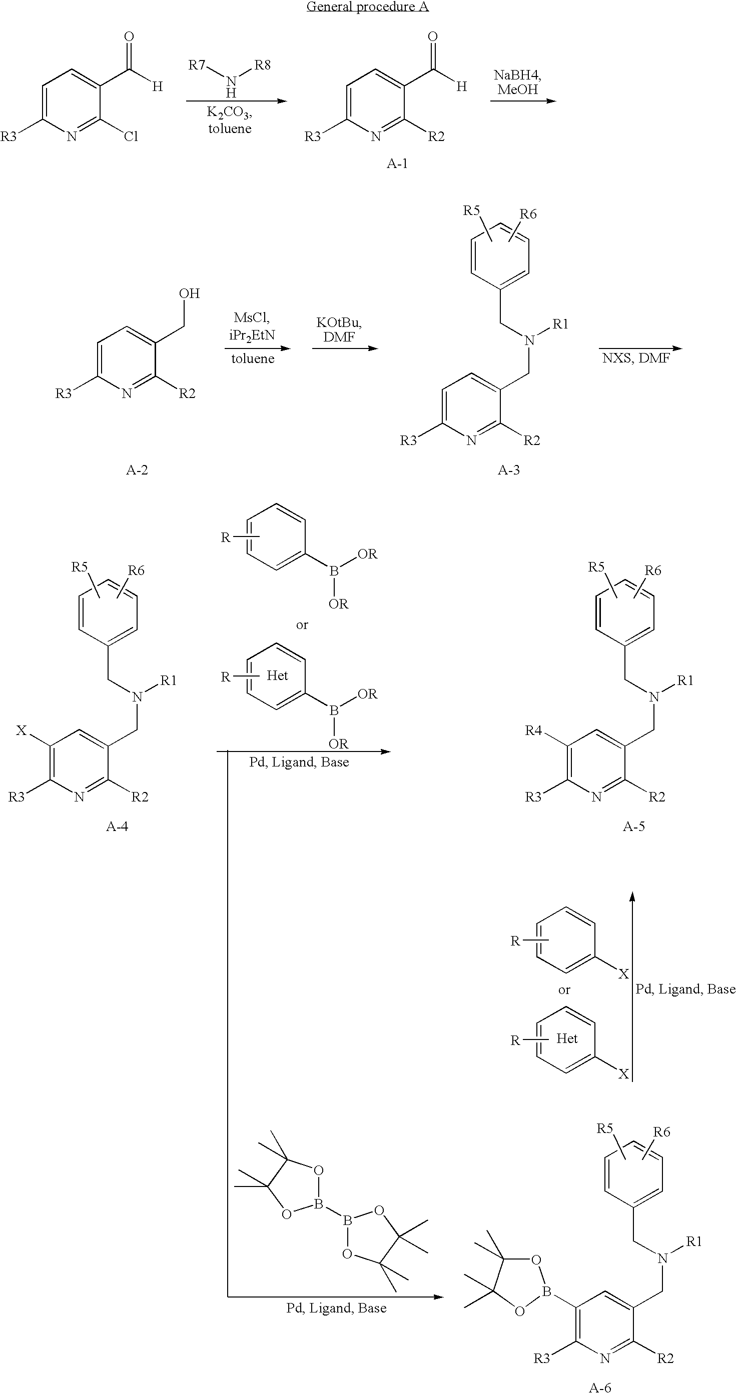

The synthesis of compounds, A-5, can be performed according to the procedures shown in Scheme A beginning either from commercially available 2-chloro-pyridine-3-carbaldehyde (CAS: 36404-88-3). The starting material can be reacted with appropriately substituted amines using base such as K₂CO₃ and Na₂CO₃ in a suitable solvent such as toluene to give compound A-1. The resulting compound A-1 can be treated with a reducing agent such as NaBH₄ in a suitable solvent such as MeOH to give compound A-2. The compound A-2 can be next condensed with appropriately substituted amines using base such as KOtBu in a suitable solvent such as DMF after treatment with MsCl and base such as iPr₂EtN in a suitable solvent such as toluene to give compound A-3. Compound A-4 can be obtained by the harogenation of the compound A-3 by N-harogenosuccinimide.

The compound A-4 can be coupled with appropriately substituted boronic acids or esters using Pd catalysis with Lignad such as Pd(PPh₃)₄, PdCl₂(dppf)₂ and FibreCat®1001 (CAS: 457645-05-5) in the presence of base such as NaOtBu and Na₂CO₃ in a suitable solvent such as toluene and EtOH to give compound A-5. Alternatively, compound A-5 can be prepared from compound A-6 and corresponding appropriately substituted halo aromatic and hetero aromatic derivatives via Pd mediated coupling reaction similar to the above reaction.

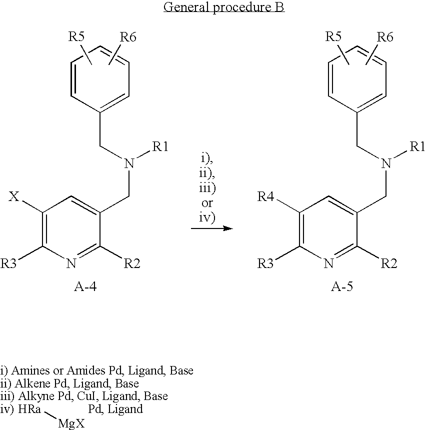

Compound A-4 can be coupled with appropriately substituted amines and esters using Pd catalysis with Lignad such as Pd₂(dba)₃ with 2-(di-tert-butylphosphino)biphenyl (CAS: 224311-51-7) or rac-2,2′-bis(diphenylphosphino)-1,1′-binaphthyl (CAS: 98327-87-8) in the presence of base such as NaOtBu in a suitable solvent such as toluene.

Compound A-4 can be converted with appropriately substituted alkenes using Pd catalysis with Ligand such as Pd₂(dba)₃ with tris(o-tolyl)phosphine (CAS: 6163-58-2) in the presence of base such as Et₃N in a suitable solvent such as DMF, or with appropriately substituted alkynes using Pd catalysis with Ligand such as PdCl₂(PPh₃)₂ and Pd(PPh₃)₄ in the presence of CuI and base such as Et₃N and iPr₂NH. Compound A-4 can be also coupled with appropriately substituted Grignard reagents using Pd catalysis such as PdCl₂(dppf)₂ in a suitable solvent such THF to give compound A-5.

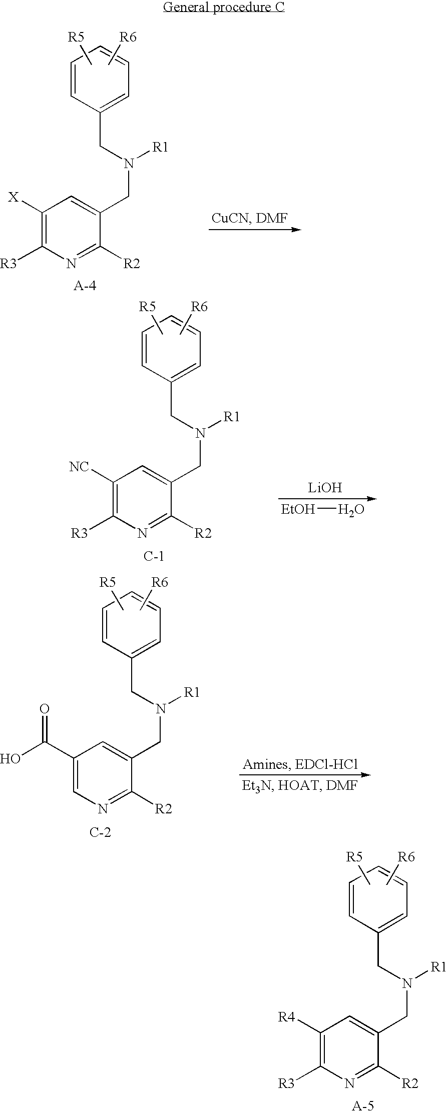

As shown in scheme C depicted above, compound A-4 can be reacted with CuCN in a suitable solvent such as DMF to give compound C-1. The resulting compound C-1 can be hydrolyzed with base such as LiOH, NaOH and KOH in a suitable solvent system such as MeOH/H₂O to give compound C-2. Compound C-2 can be next condensed with appropriately substituted amines using a coupling reagent such as 1-ethyl-3-(3-dimethylaminopropyl)carbodiimide hydrochloride in the presence of hydroxy-7-azabenzotriazole and base such as Et₃N in a suitable solvent such as DMF to give compound A-5.

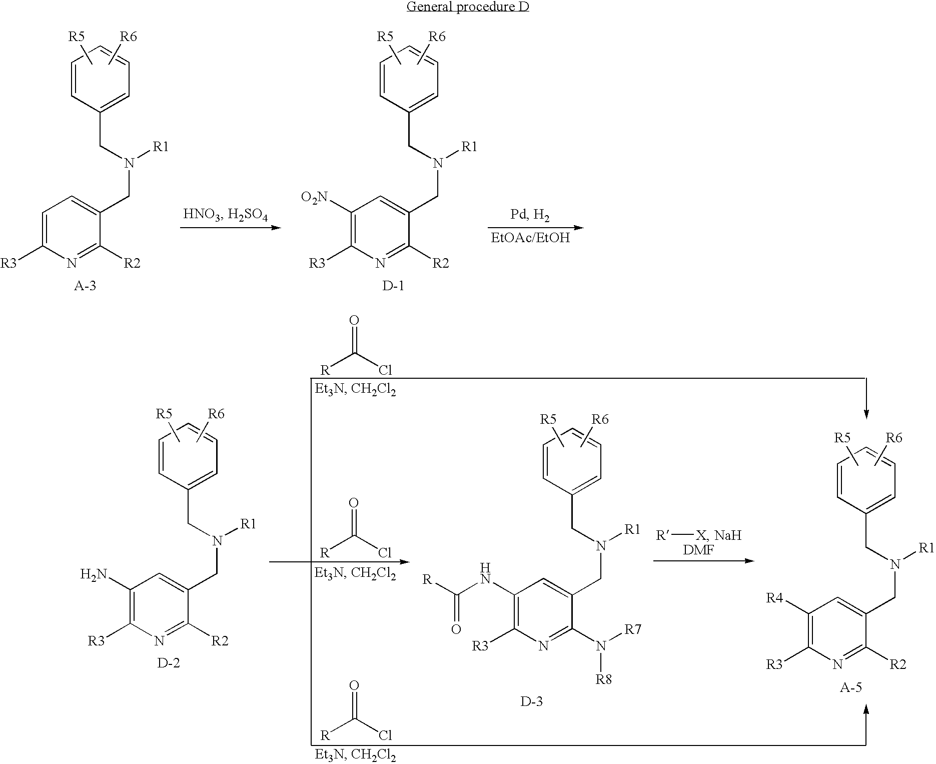

Compound A-3 can be treated with HNO₃ (fuming) in H₂SO₄ to give compound D-1. Nitro group of the compound D-1 can be reduced using Pd catalysis under H₂ atmosphere to give compound D-2. The resulting compound D-2 can be next condensed with appropriately substituted acid chlorides or sulfonyl chlorides in the presence of base such as Et₃N in a suitable solvent such as CH₂Cl₂ to give compound D-3 or A-5. Compound A-5 can be obtained by alkylation of the compound D-3 with appropriately substituted alkyl halides in the presence of base such as NaH in a suitable solvent such as DMF.

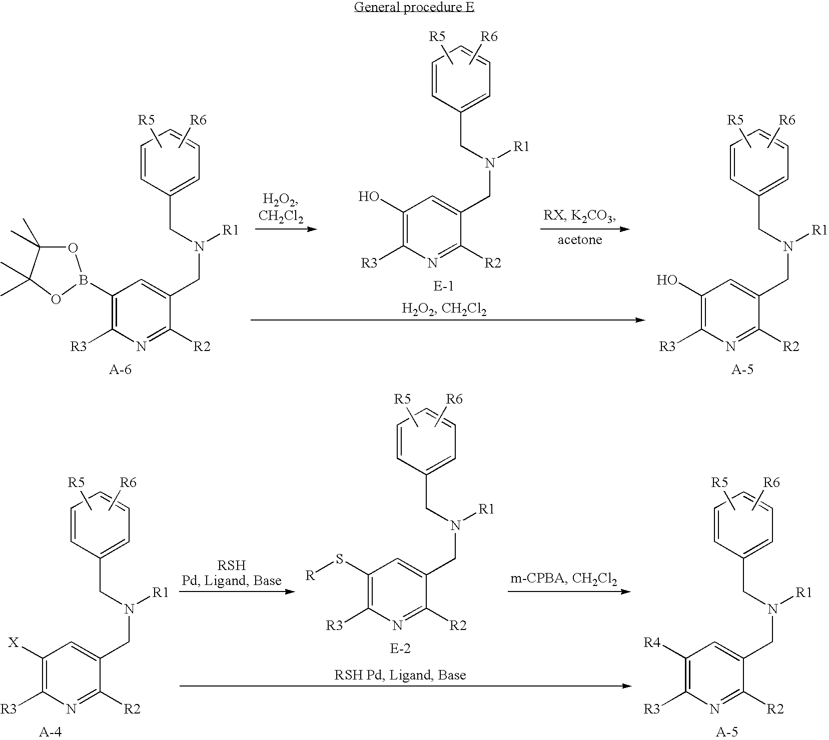

Compound A-6 can be converted to compound E-1 or compound A-5 using an oxidizing reagent such as H₂O₂ in a suitable solvent such as CH₂Cl₂. Alkylation of the compound E-1 can be performed with appropriately substituted alkyl halides in the presence of base such as K₂CO₃ in a suitable solvent such as acetone to afford compound A-5. Compound A-4 can be converted to compound E-2 or compound A-5 with appropriately substituted thiols via Pd mediated reaction. The resulting compound E-2 is treated with an oxidizing reagent such as m-CPBA in a suitable solvent such as CH₂Cl₂ to give compound A-5.

Racemates and diastereomer mixtures obtained can be separated into the pure isomers or racemates in a known manner on the basis of the physicochemical differences of the components, for example by fractional crystallization or by chiral chromatography or HPLC separation utilizing chiral stationery phases. Racemates obtained may furthermore be resolved into the optical antipodes by known methods, for example by recrystallization from an optically active solvent, chromatography on chiral adsorbents, with the aid of suitable microorganisms, by cleavage with specific immobilized enzymes, via the formation of inclusion compounds, for example using chiral crown ethers, only one enantiomer being complexed, or by conversion into diastereomeric salts, for example by reaction of a basic final substance racemate with an optically active acid, such as a carboxylic acid, for example tartaric or malic acid, or sulfonic acid, for example camphorsulfonic acid, and separation of the diastereomer mixture obtained in this manner, for example on the basis of its differing solubilities, into the diastereomers from which the desired enantiomer can be liberated by the action of suitable agents. The more active enantiomer is advantageously isolated.

In starting compounds and intermediates which are converted to the compounds of the invention in a manner described herein, functional groups present, such as amino, thiol, carboxyl and hydroxy groups, are optionally protected by conventional protecting groups that are common in preparative organic chemistry. Protected amino, thiol, carboxyl and hydroxy groups are those that can be converted under mild conditions into free amino thiol, carboxyl and hydroxy groups without the molecular framework being destroyed or other undesired side reactions taking place.

The purpose of introducing protecting groups is to protect the functional groups from undesired reactions with reaction components under the conditions used for carrying out a desired chemical transformation. The need and choice of protecting groups for a particular reaction is known to those skilled in the art and depends on the nature of the functional group to be protected (hydroxy group, amino group, etc.), the structure and stability of the molecule of which the substituent is a part and the reaction conditions.

Well-known protecting groups that meet these conditions and their introduction and removal are described, e.g., in McOmie, “Protective Groups in Organic Chemistry”, Plenum Press, London, NY (1973); and Greene and Wuts, “Protective Groups in Organic Synthesis”, John Wiley and Sons, Inc., NY (1999).

The above-mentioned reactions are carried out according to standard methods, in the presence or absence of diluent, preferably, such as are inert to the reagents and are solvents thereof, of catalysts, condensing or said other agents, respectively and/or inert atmospheres, at low temperatures, room temperature or elevated temperatures, preferably at or near the boiling point of the solvents used, and at atmospheric or super-atmospheric pressure. The preferred solvents, catalysts and reaction conditions are set forth in the appended illustrative Examples.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known per se.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers etc.

Preferably, the pharmaceutical compositions are tablets and gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable compositions for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

The present invention further provides anhydrous pharmaceutical compositions and dosage forms comprising the compounds of the present invention as active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The invention likewise relates to a combination of a compound of formula (I), (I A) or (I B), respectively, or a pharmaceutically acceptable salt thereof with a further active principle.

The combination may be made for example with the following active principles, selected from the group consisting of a:

(i) HMG-Co-A reductase inhibitor or a pharmaceutically acceptable salt thereof,

(ii) angiotensin II receptor antagonist or a pharmaceutically acceptable salt thereof,

(iii) angiotensin converting enzyme (ACE) Inhibitor or a pharmaceutically acceptable salt thereof,

(iv) calcium channel blocker or a pharmaceutically acceptable salt thereof,

(v) aldosterone synthase inhibitor or a pharmaceutically acceptable salt thereof,

(vi) aldosterone antagonist or a pharmaceutically acceptable salt thereof,

(vii) dual angiotensin converting enzyme/neutral endopeptidase (ACE/NEP) inhibitor or a pharmaceutically acceptable salt thereof,

(viii) endothelin antagonist or a pharmaceutically acceptable salt thereof,

(ix) renin inhibitor or a pharmaceutically acceptable salt thereof,

(x) diuretic or a pharmaceutically acceptable salt thereof, and

(xi) an ApoA-I mimic.

An angiotensin II receptor antagonist or a pharmaceutically acceptable salt thereof is understood to be an active ingredients which bind to the AT₁-receptor subtype of angiotensin II receptor but do not result in activation of the receptor. As a consequence of the inhibition of the AT₁ receptor, these antagonists can, for example, be employed as antihypertensives or for treating congestive heart failure.

The class of AT₁ receptor antagonists comprises compounds having differing structural features, essentially preferred are the non-peptidic ones. For example, mention may be made of the compounds which are selected from the group consisting of valsartan, losartan, candesartan, eprosartan, irbesartan, saprisartan, tasosartan, telmisartan, the compound with the designation E-1477 of the following formula

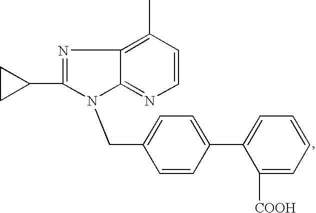

the compound with the designation SC-52458 of the following formula

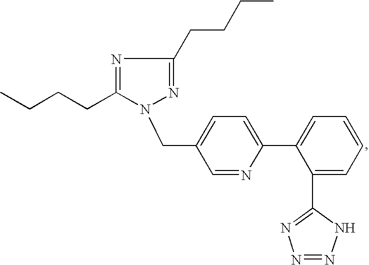

and the compound with the designation ZD-8731 of the following formula

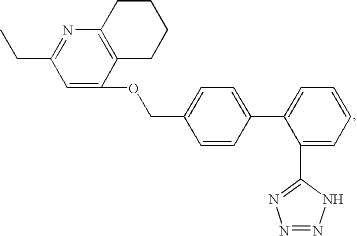

or, in each case, a pharmaceutically acceptable salt thereof.

Preferred AT₁-receptor antagonist are those agents which have been marketed, most preferred is valsartan or a pharmaceutically acceptable salt thereof.

HMG-Co-A reductase inhibitors (also called □-hydroxy-□-methylglutaryl-co-enzyme-A reductase inhibitors) are understood to be those active agents that may be used to lower the lipid levels including cholesterol in blood.

The class of HMG-Co-A reductase inhibitors comprises compounds having differing structural features. For example, mention may be made of the compounds that are selected from the group consisting of atorvastatin, cerivastatin, compactin, dalvastatin, dihydrocompactin, fluindostatin, fluvastatin, lovastatin, pravastatin, mevastatin, pravastatin, rivastatin, simvastatin, and velostatin, or, in each case, a pharmaceutically acceptable salt thereof.

Preferred HMG-Co-A reductase inhibitors are those agents which have been marketed, most preferred is fluvastatin and pitavastatin or, in each case, a pharmaceutically acceptable salt thereof.

The interruption of the enzymatic degradation of angiotensin I to angiotensin II with so-called ACE-inhibitors (also called angiotensin converting enzyme inhibitors) is a successful variant for the regulation of blood pressure and thus also makes available a therapeutic method for the treatment of congestive heart failure.

The class of ACE inhibitors comprises compounds having differing structural features. For example, mention may be made of the compounds which are selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, in each case, a pharmaceutically acceptable salt thereof.

Preferred ACE inhibitors are those agents that have been marketed, most preferred are benazepril and enalapril.

The class of CCBs essentially comprises dihydropyridines (DHPs) and non-DHPs such as diltiazem-type and verapamil-type CCBs.

A CCB useful in said combination is preferably a DHP representative selected from the group consisting of amlodipine, felodipine, ryosidine, isradipine, lacidipine, nicardipine, nifedipine, niguldipine, niludipine, nimodipine, nisoldipine, nitrendipine, and nivaldipine, and is preferably a non-DHP representative selected from the group consisting of flunarizine, prenylamine, diltiazem, fendiline, gallopamil, mibefradil, anipamil, tiapamil and verapamil, and in each case, a pharmaceutically acceptable salt thereof. All these CCBs are therapeutically used, e.g. as anti-hypertensive, anti-angina pectoris or anti-arrhythmic drugs.

Preferred CCBs comprise amlodipine, diltiazem, isradipine, nicardipine, nifedipine, nimodipine, nisoldipine, nitrendipine, and verapamil, or, e.g. dependent on the specific CCB, a pharmaceutically acceptable salt thereof. Especially preferred as DHP is amlodipine or a pharmaceutically acceptable salt, especially the besylate, thereof. An especially preferred representative of non-DHPs is verapamil or a pharmaceutically acceptable salt, especially the hydrochloride, thereof.

Aldosterone synthase inhibitor is an enzyme that converts corticosterone to aldosterone to by hydroxylating cortocosterone to form 18-OH-corticosterone and 18-OH-corticosterone to aldosterone. The class of aldosterone synthase inhibitors is known to be applied for the treatment of hypertension and primary aldosteronism comprises both steroidal and non-steroidal aldosterone synthase inhibitors, the later being most preferred.

Preference is given to commercially available aldosterone synthase inhibitors or those aldosterone synthase inhibitors that have been approved by the health authorities.

The class of aldosterone synthase inhibitors comprises compounds having differing structural features. For example, mention may be made of the compounds which are selected from the group consisting of the non-steroidal aromatase inhibitors anastrozole, fadrozole (including the (+)-enantiomer thereof), as well as the steroidal aromatase inhibitor exemestane, or, in each case where applicable, a pharmaceutically acceptable salt thereof.

The most preferred non-steroidal aldosterone synthase inhibitor is the (+)-enantiomer of the hydrochloride of fadrozole (U.S. Pat. Nos. 4,617,307 and 4,889,861) of formula

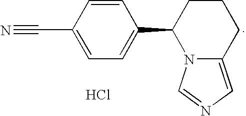

A preferred steroidal aldosterone antagonist is eplerenone of the formula

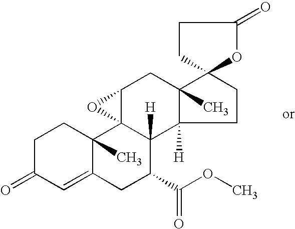

spironolactone.

A preferred dual angiotensin converting enzyme/neutral endopetidase (ACE/NEP) inhibitor is, for example, omapatrilate (cf. EP 629627), fasidotril or fasidotrilate, or, if appropriable, a pharmaceutically acceptable salt thereof.

A preferred endothelin antagonist is, for example, bosentan (cf. EP 526708 A), furthermore, tezosentan (cf. WO 96/19459), or in each case, a pharmaceutically acceptable salt thereof.

A renin inhibitor is, for example, a non-peptidic renin inhibitor such as the compound of formula

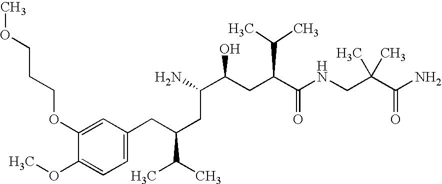

chemically defined as 2(S),4(S),5(S),7(S)-N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide. This representative is specifically disclosed in EP 678503A. Especially preferred is the hemi-fumarate salt thereof.

A diuretic is, for example, a thiazide derivative selected from the group consisting of chlorothiazide, hydrochlorothiazide, methylclothiazide, and chlorothalidon. The most preferred is hydrochlorothiazide.

An ApoA-I mimic is, for example, D4F peptide, especially of formula D-W-F-K-A-F-Y-D-K-V-A-E-K-F-K-E-A-F

Preferably, the jointly therapeutically effective amounts of the active agents according to the combination of the present invention can be administered simultaneously or sequentially in any order, separately or in a fixed combination.

The structure of the active agents identified by generic or tradenames may be taken from the actual edition of the standard compendium “The Merck Index” or from databases, e.g. IMS LifeCycle (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

Furthermore, the combinations as described above can be administered to a subject via simultaneous, separate or sequential administration (use). Simultaneous administration (use) can take place in the form of one fixed combination with two or more active ingredients, or by simultaneously administering two or more compounds that are formulated independently. Sequential administration (use) preferably means administration of one (or more) compounds or active ingredients of a combination at one time point, other compounds or active ingredients at a different time point, that is, in a chronically staggered manner, preferably such that the combination shows more efficiency than the single compounds administered independently (especially showing synergism). Separate administration (use) preferably means administration of the compounds or active ingredients of the combination independently of each other at different time points, preferably meaning that two compounds are administered such that no overlap of measurable blood levels of both compounds are present in an overlapping manner (at the same time).

Also combinations of two or more of sequential, separate and simultaneous administrations are possible, preferably such that the combination compound-drugs show a joint therapeutic effect that exceeds the effect found when the combination compound-drugs are used independently at time intervals so large that no mutual effect on their therapeutic efficiency can be found, a synergistic effect being especially preferred.

Additionally, the present invention provides:

a pharmaceutical composition or combination of the present invention for use as a medicament;

the use of a pharmaceutical composition or combination of the present invention for the delay of progression and/or treatment of a disorder or disease mediated by CETP or responsive to the inhibition of CETP.

the use of a pharmaceutical composition or combination of the present invention for the delay of progression and/or treatment of a disorder or disease selected from hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity or endotoxemia etc.

The pharmaceutical composition or combination of the present invention can be in unit dosage of about 1-1000 mg of active ingredients for a subject of about 50-70 kg, preferably about 5-500 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, preferably between about 1-100 mg/kg.

The CETP inhibitory effect of the compounds of the present invention can be determined by using the test models or assays known in the art. For example, EP1115695B1 describes both the in vitro and in vivo CETP activity assays, the contents of which are hereby incorporated by reference. In particular, the following assays are used.

For purposes of detecting the in vitro and in vivo CETP activity, human pro-apolipoprotein AI (pro-apoAI) and donor microemulsion are prepared as follows. The cDNA of human pro-apoAI (NCBI accession number: NM_{—}000039) is cloned from human liver Quick-Clone™ cDNA (Clontech, CA) and inserted to a pET28a vector (Novagen, Germany) for bacterial expression. Expressed protein as a fusion protein with 6×His-tag at N-terminus in BL-21 Gold (DE3) (Strategene, CA) is purified using HiTrap Chelating (GE Healthcare, CT). Pro-apoAI containing microemulsion as a donor particle is prepared following previous reports (J. Biol. Chem., 280:14918-22). Glyceryl trioleate (62.5 ng, Sigma, Mo.), 3-sn-phosphatidylcholine (583 ng, Wako Pure Chemical Industries, Japan), and cholesteryl BODIPY® FL C₁₂ (250 ng, Invitrogen, CA) are dissolved in 1 mL of chloroform. The solution is evaporated, then residual solvent is removed in vacuum for more than 1 hr. The dried lipid mixture is dissolved in 500 μL of the assay buffer (50 mM Tris-HCl (pH7.4) containing 150 mM NaCl and 2 mM EDTA) and sonicated at 50° C. with a microtip (MICROSON™ ULTRASONIC CELL DISRUPTOR, Misonix, Farmingdale, N.Y.) at output power 006 for 2 min. After sonication, the solution is cooled to 40° C., added to 100 μg of human pro-apoAI, and sonicated at output power 004 for 5 min at 40° C. The solution, BODIPY-CE microemulsion as a donor molecule is stored at 4° C. after filtration through a 0.45 μm PVDF filter.

For purposes of detecting in vitro CETP activity, human EDTA plasma samples from healthy men are purchased from New Drug Development Research Center, Inc. Donor solution is prepared by a dilution of donor microemulsion with assay buffer. Human plasma (50 μL), assay buffer (35 μL) and test compound dissolved in dimethylsulfoxide (1 μL) are added to each well of 96 well half area black flat bottom plate. The reaction is started by the addition of donor solution (14 μL) into each well. Fluorescence intensities are measured every 30 min at 37° C. with excitation wave length of 485 nm and emission wavelength of 535 nm. The CETP activity (Fl/min) is defined as the changes of fluorescence intensity from 30 to 90 min. The IC₅₀ value is obtained by the logistic equation (Y=Bottom+(Top-Bottom)/(1+(x/IC₅₀)̂Hill slope) using Origin software, version 7.5 SR3.

Effects of compounds on HDL-cholesterol level in hamsters are investigated by the method reported previously with some modifications (Eur. J. Phamacol., 466 (2003) 147-154). In brief, male Syrian hamsters (SLC, Shizuoka, Japan) are fed a high cholesterol diet for two weeks. Then, the animals are dosed singly with a compound suspended in 5% methyl cellulose solution. HDL-cholesterol levels are measured by using commercially available kit (Wako Pure Chemical, Japan) after the precipitation of apolipoprotein B (apoB)-containing lipoproteins with 13% polyethylene glycol 6000.

To detect the ex vivo CETP activity, heparin plasma samples are prepared from compound treated hamsters. Donor solution is prepared by a dilution of donor microemulsion with assay buffer. Hamster plasma (50 μL) and donor solution (10 μL) are added to each well of 96 well half area black flat bottom plate. Then fluorescence intensities are measured every 15 min at 37° C. with excitation wave length of 485 nm and emission wavelength of 535 nm. The CETP activity (Fl/min) is defined as the changes of fluorescence intensity from 30 to 90 min. The potency of a compound is calculated as a % inhibition of the CETP activity in vehicle treated hamster plasma.

TABLE 1
Inhibitory Activity of Compounds

| | | CETP Plasma |
|---|---|---|
| # | Compound | IC₅₀ (nM) |
| 1 | (5-Azetidin-1-yl-3-{[(3,5-bis-trifluoromethyl-benzyl)-(2- | 260 |
| | methyl-2H-tetrazol-5-yl)-amino]-methyl}-pyridin-2-yl)- | |
| | cyclopentylmethyl-ethyl-amine | |
| 2 | N-[5-{[(3,5-Bis-trifluoromethyl-benzyl)-(2-methyl-2H- | 370 |
| | tetrazol-5-yl)-amino]-methyl}-6-(cyclopentylmethyl- | |
| | ethyl-amino)-pyridin-3-yl]-N-methyl-acetamide | |
| 3 | (3-{[(3,5-Bis-trifluoromethyl-benzyl)-(2-methyl-2H- | 140 |
| | tetrazol-5-yl)-amino]-methyl}-5-methylsulfanyl-pyridin- | |
| | 2-yl)-cyclopentylmethyl-ethyl-amine | |
| 4 | (3-{[(3,5-Bis-trifluoromethyl-benzyl)-(2-methyl-2H- | 110 |
| | tetrazol-5-yl)-amino]-methyl}-5-bromo-6-methyl-pyridin- | |
| | 2-yl)-cyclopentylmethyl-ethyl-amine | |
| 5 | (3-{[(3,5-Bis-trifluoromethyl-benzyl)-(2-methyl-2H- | 300 |
| | tetrazol-5-yl)-amino]-methyl}-5-methyl-pyridin-2-yl)- | |
| | cyclopentylmethyl-ethyl-amine | |
| 6 | (3-{[(3,5-Bis-trifluoromethyl-benzyl)-(2-methyl-2H- | 230 |
| | tetrazol-5-yl)-amino]-methyl}-5-iodo-pyridin-2-yl)- | |
| | cyclopentylmethyl-ethyl-amine | |
| 7 | 3-{[(3,5-Bis-trifluoromethyl-benzyl)-(2-methyl-2H- | 720 |
| | tetrazol-5-yl)-amino]-methyl}-N*5*-butyl-N*2*- | |
| | cyclopentylmethyl-N*2*-ethyl-N*5*-methyl-pyridine-2,5- | |
| | diamine | |
| 8 | (3-{[(3,5-Bis-trifluoromethyl-benzyl)-(2-methyl-2H- | 630 |
| | tetrazol-5-yl)-amino]-methyl}-5-nitro-pyridin-2-yl)- | |
| | cyclopentylmethyl-ethyl-amine | |

### ABBREVIATIONS

### Ac: Acetyl

dba:dibenzylidenacetone

### DMAP: N,N-dimethylaminopyridine

DME: dimethoxyethane

### DMF: N,N-dimethylformamide

dppf: 1,1-bis(diphenylphosphino)ferrocene
ESI: electrospray ionization
EtOAc: ethyl acetate
h: hours
HPLC: high pressure liquid chromatography
IPA: 2-propanol
iPr: isopropyl
LC: liquid chromatography
LHMDS: lithium hexamethyldisilamide
min: minutes
MS: mass spectrometry
NMR: nuclear magnetic resonance
THF: tetrahydrofuran
tol: tolyl

### EXAMPLES

The following examples are intended to illustrate the invention and are not to be construed as being limitations thereon. Temperatures are given in degrees centrigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art. The compounds in the following examples have been found to have IC₅₀ values in the range of about 0.1 nM to about 100,0.00 nM for CETP.

### Example 1

### Synthesis of 3-{[(3,5-bistrifluoromethylbenzyl)(2-methyl-2H-tetrazol-5-yl)amino]methyl}-5-(2-methylthiophen-3-yl)pyridin-2-yl]cyclopentylmethylethylamine

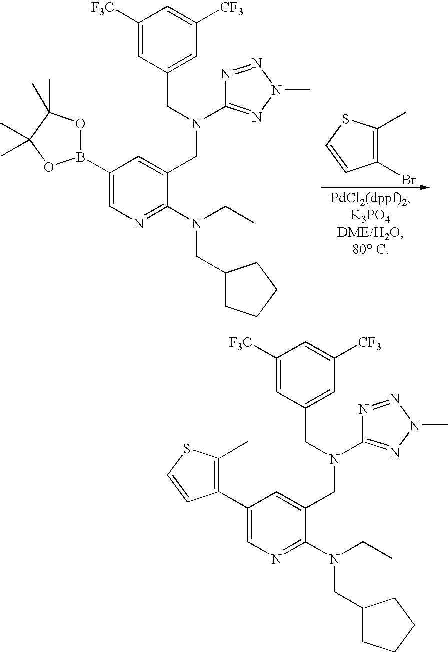

A mixture of 3-{[(3,5-bistrifluoromethylbenzyl)(2-methyl-2H-tetrazol-5-yl)amino]methyl}-5-(4,4,5,5-tetramethyl[1,3,2]dioxaborolan-2-yl)pyridin-2-yl]cyclopentylmethylethylamine (110 mg, 0.16 mmol), K₃PO₄ (105 mg, 0.50 mmol), 2-Methyl-3-bromothiophene (44 mg, 0.24 mmol CAS: 30319-05-2) and PdCl₂(dppf)₂ (13 mg, 0.016 mmol) in DME/H₂O (10:1, 1.7 mL) is heated at 80° C. overnight. After cooling down to room temperature, H₂O is added to the reaction mixture. The mixture is extracted with EtOAc. The organic layer is washed with H₂O, dried and concentrated under reduced pressure. The resulting residue is purified by silica gel flash chromatography to give 3-{[(3,5-bistrifluoromethylbenzyl)(2-methyl-2H-tetrazol-5-yl)amino]methyl}-5-(4,4,5,5-tetramethyl[1,3,2]dioxaborolan-2-yl)pyridin-2-yl]cyclopentylmethylethylamine as pale yellow oil (756 mg, 0.88 mmol; 98%); ESI-MS m/z: 638 [M+1]⁺, Retention time 2.12 min.

### Example 2

### [3-{[(3,5-bistrifluoromethylbenzyl)(2-methyl-2H-tetrazol-5-yl)amino]methyl}-5-(4-methylthiophen-3-yl)pyridin-2-yl]cyclopentylmethylethylamine

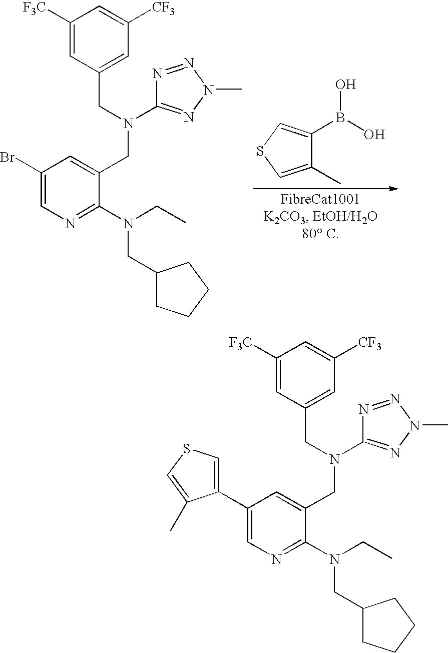

A mixture of (3-{[(3,5-bistrifluoromethylbenzyl)(2-methyl-2H-tetrazol-5-yl)amino]methyl}-5-bromopyridin-2-yl)cyclopentylmethylethylamine (156 mg, 0.24 mmol), K₂CO₃ (52 mg, 0.38 mmol) and FibreCat®1001 (77 mg, 0.024 mmol, CAS: 457645-05-5) in EtOH/H₂O (10:1, 1.2 mL) is heated at 80° C. overnight. After cooling down to room temperature, the reaction mixture is filtered. H₂O is added to the mixture. The mixture is extracted with EtOAc. The filtrate is purified by silica gel flash chromatography to give [3-{[(3,5-bistrifluoromethylbenzyl)(2-methyl-2H-tetrazol-5-yl)amino]methyl}-5-(4-methylthiophen-3-yl)pyridin-2-yl]cyclopentylmethylethylamine as pale yellow oil (93 mg, 0.089 mmol; 39%); ESI-MS m/z: 638 [M+1]⁺, Retention time: 2.12 min.

### Example 3

The following compounds are prepared following the procedure of Example 1-2.

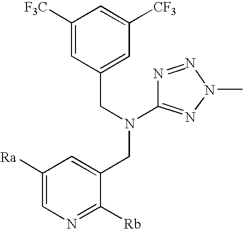

| | | | | LC |
|---|---|---|---|---|
| No. | Ra | Rb | MS | (min) |
| 1 | 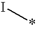 | 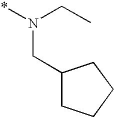 | 668 [M + 1]⁺ | 2.45 |
| 2 | 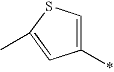 | 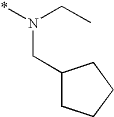 | 638 [M + 1]⁺ | 2.13 |
| 3 | 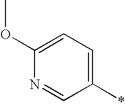 | 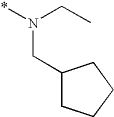 | 649 [M + 1]⁺ | 2.08 |
| 4 | 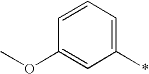 | 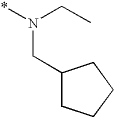 | 648 [M + 1]⁺ | 2.10 |
| 5 | 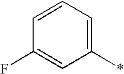 | 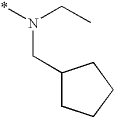 | 636 [M + 1]⁺ | 2.14 |

### Example 4

### Synthesis of (5′-{[(3,5-bistrifluoromethylbenzyl)(2-methyl-2H-tetrazol-5-yl)amino]methyl}-3,4,5,6-tetrahydro-2H-[1,3′]bipyridinyl-6′-yl)cyclopentylmethylethylamine

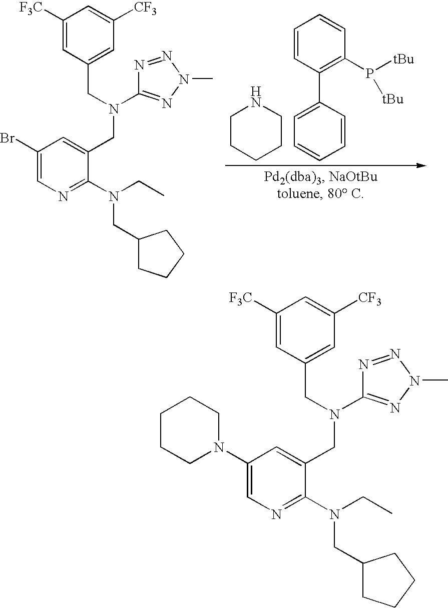

A mixture of (3-{[(3,5-bistrifluoromethylbenzyl)(2-methyl-2H-tetrazol-5-yl)amino]methyl}-5-bromopyridin-2-yl)cyclopentylmethylethylamine (130 mg, 0.21 mmol), sodium tert-butoxide (30 mg, 0.31 mmol), piperidine (27 mg, 0.31 mmol), Pd₂(dba)₃ (19 mg, 0.021 mmol,) and 2-(di-tert-butylphosphino)biphenyl (6.3 mg, 0.021 mmol, CAS: 224311-51-7) in toluene (1.0 mL) is heated at 80° C. for 2 h. Silica gel is added to the reaction mixture. After filtration, the filtrate is concentrated under reduced pressure. The resulting residue is purified by reverse phase preparative HPLC to give (5′-{[(3,5-bistrifluoromethylbenzyl)(2-methyl-2H-tetrazol-5-yl)amino]methyl}-3,4,5,6-tetrahydro-2H-[1,3′]bipyridinyl-6′-yl)cyclopentylmethylethylamine as pale yellow oil (71 mg, 0.11 mmol; 54%); ESI-MS m/z: 625 [M+1]⁺, Retention time: 2.04 min.

### Example 5

### Synthesis of 1-[5-{[(3,5-bistrifluoromethylbenzyl)-(2-methyl-2H-tetrazol-5-yl)amino]methyl}-6-(cyclopentylmethylethylamino)pyridin-3-yl]pyrrolidin-2-one

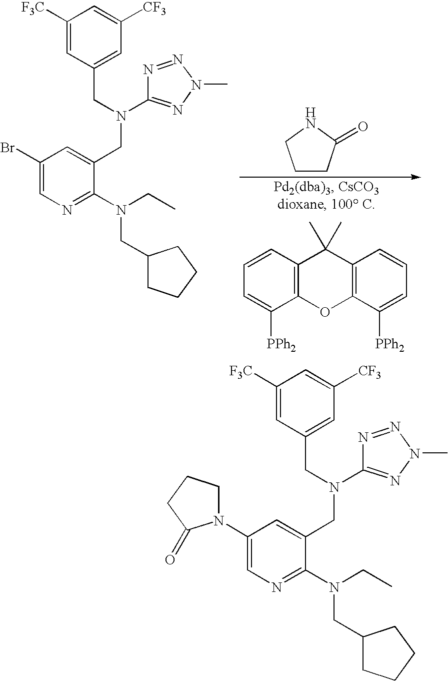

A mixture of (3-{[(3,5-bistrifluoromethylbenzyl)(2-methyl-2H-tetrazol-5-yl)amino]methyl}-5-bromopyridin-2-yl)cyclopentylmethylethylamine (136 mg, 0.22 mmol), cesium carbonate (107 mg, 0.33 mmol), 2-pyrrolidinone (28 mg, 0.33 mmol), Pd₂(dba)₃ (10 mg, 0.011 mmol,) and 2-4,5-bis(diphenylphosphino)-9,9-dimethylxanthene (13 mg, 0.022 mmol, CAS: 161265-03-8) in dioxane (1.0 mL) is heated at 100° C. overnight. The reaction mixture is cooled down to room temperature and filtered through Cerite® with EtOAc. The filtrate is washed with brine, dried and concentrated under reduced pressure. The resulting residue is purified by reverse phase preparative HPLC to give 1-[5-{[(3,5-bistrifluoromethylbenzyl)-(2-methyl-2H-tetrazol-5-yl)amino]methyl}-6-(cyclopentylmethylethylamino)pyridin-3-yl]pyrrolidin-2-one as pale yellow oil (12 mg, 0.019 mmol; 9%); ESI-MS m/z: 625 [M+1]⁺, Retention time: 1.99 min.

### Example 6

### Synthesis of 3-{[(3,5-bistrifluoromethylbenzyl)(2-methyl-2H-tetrazol-5-yl)amino]methyl}-N*2*-cyclopentylmethyl-N*2*-ethyl-N*5*-isobutylpyridine-2,5-diamine

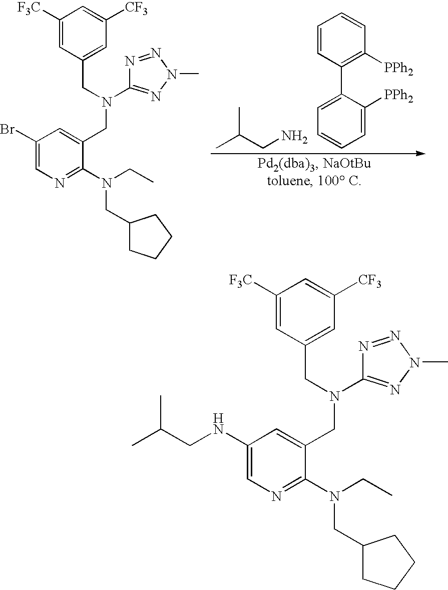

A mixture of (3-{[(3,5-bistrifluoromethylbenzyl)(2-methyl-2H-tetrazol-5-yl)amino]methyl}-5-bromopyridin-2-yl)cyclopentylmethylethylamine (134 mg, 0.22 mmol), sodium tert-butoxide (31 mg, 0.33 mmol), isobutylamine (24 mg, 0.33 mmol), Pd₂(dba)₃ (20 mg, 0.022 mmol,) and rac-2,2′-bis(diphenylphosphino)-1,1′-binaphthyl (13 mg, 0.022 mmol, CAS: 98327-87-8) in toluene (1.0 mL) is heated at 100° C. overnight. The reaction mixture is cooled down to room temperature and filtered through Cerite® with EtOAc. The filtrate is washed with H₂O, dried and concentrated under reduced pressure. The resulting residue is purified by reverse phase preparative HPLC to give 3-{[(3,5-bistrifluoromethylbenzyl)(2-methyl-2H-tetrazol-5-yl)amino]methyl}-N*2*-cyclopentylmethyl-N*2*-ethyl-N*5*-isobutylpyridine-2,5-diamine as pale yellow oil (50 mg, 0.082 mmol; 38%); ESI-MS m/z: 613[M+1]⁺, Retention time: 2.06 min.

### Example 7

The following compounds are prepared following the procedure of Example 4-6.

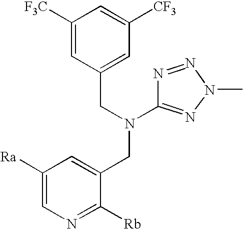

| | | | | LC |
|---|---|---|---|---|
| No. | Ra | Rb | MS | (min) |
| 1 | 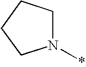 | 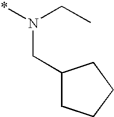 | 611 [M + 1]⁺ | 2.05 |
| 2 | 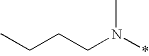 | 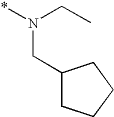 | 627 [M + 1]⁺ | 2.08 |
| 3 | 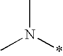 | 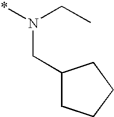 | 585 [M + 1]⁺ | 2.08 |
| 4 | 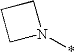 | 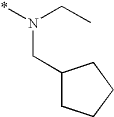 | 597 [M + 1]⁺ | 2.03 |
| 5 | 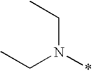 | 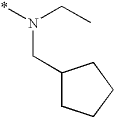 | 613 [M + 1]⁺ | 2.08 |
| 6 | 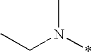 | 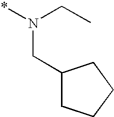 | 599 [M + 1]⁺ | 2.03 |
| 7 | 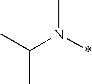 | 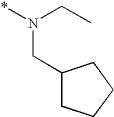 | 613 [M + 1]⁺ | 2.08 |

### Example 8

### Synthesis of (E)-3-[5-{[(3,5-bistrifluoromethylbenzyl)(2-methyl-2H-tetrazol-5-yl)amino]methyl}-6-(cyclopentylmethylethylamino)pyridin-3-yl]acrylic acid methyl ester

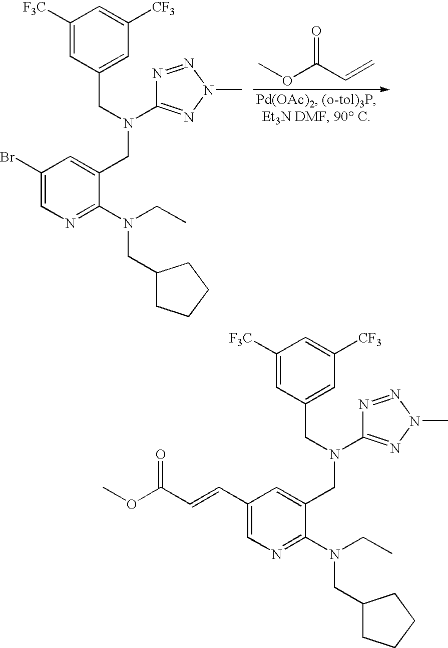

A mixture of (3-{[(3,5-bistrifluoromethylbenzyl)(2-methyl-2H-tetrazol-5-yl)amino]methyl}-5-bromopyridin-2-yl)cyclopentylmethylethylamine (155 mg, 0.25 mmol), Et₃N (51 mg, 0.50 mmol), acrylic acid methyl ester (43 mg, 0.50 mmol), Pd(OAc)₂ (6.0 mg, 0.050 mmol,) and tris(o-tolyl)phosphine (8.0 mg, 0.050 mmol, CAS: 6163-58-2) in DMF (1.0 mL) is heated at 90° C. overnight. The reaction mixture is cooled down to room temperature. After addition of H₂O, the reaction mixture is extracted with CH₂Cl₂. The organic layer is dried and concentrated under reduced pressure. The resulting residue is purified by reverse phase preparative HPLC to give (E)-3-[5-{[(3,5-bistrifluoromethylbenzyl)(2-methyl-2H-tetrazol-5-yl)amino]methyl}-6-(cyclopentylmethylethylamino)pyridin-3-yl]acrylic acid methyl ester yellow oil (32 mg, 0.051 mmol; 20%); ESI-MS m/z: 626 [M+1]⁺, Retention time: 2.13 min.

### Example 9

### Synthesis of (3-{[(3,5-bistrifluoromethylbenzyl)(2-methyl-2H-tetrazol-5-yl)-amino]methyl}-5-trimethylsilanylethynylpyridin-2-yl)cyclopentylmethylethylamine

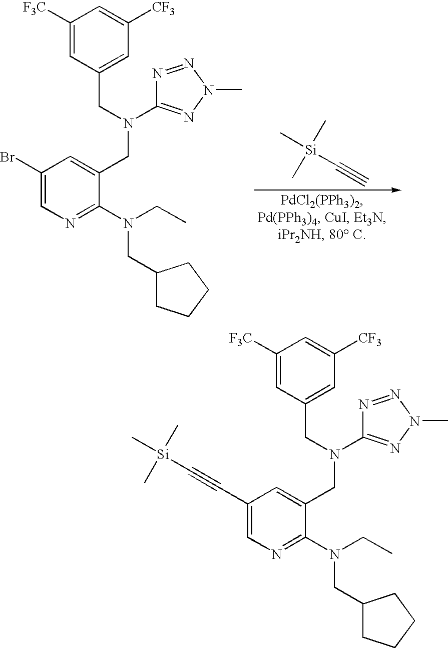

A mixture of (3-{[(3,5-bistrifluoromethylbenzyl)(2-methyl-2H-tetrazol-5-yl)amino]methyl}-5-bromopyridin-2-yl)cyclopentylmethylethylamine (155 mg, 0.25 mmol), trimethylsilyl acetylene (32 mg, 0.33 mmol), PdCl₂(PPh₃)₂ (15 mg, 0.021 mmol) and CuI (4.0 mg, 0.021 mmol) in Et₃N (1.5 mL) is heated at 80° C. overnight. To the reaction mixture, are added Pd(PPh₃)₄ (25 mg, 0.021 mmol) and iPr₂NH (1.0 mL). After stirring at 80° C. for 8 h, H₂O is added to the reaction mixture at room temperature. The mixture is extracted with EtOAc. The organic layer is dried and concentrated under reduced pressure. The resulting residue purified by silica gel flash chromatography to give (3-{[(3,5-bistrifluoromethylbenzyl)(2-methyl-2H-tetrazol-5-yl)-amino]methyl}-5-trimethylsilanylethynylpyridin-2-yl)cyclopentylmethylethylamine as colorless oil (96 mg, 0.14 mmol; 70%); ESI-MS m/z: 638 [M+1]⁺, Retention time: 2.48 min.

### Example 10

### Synthesis (3-{[(3,5-bistrifluoromethylbenzyl)-(2-methyl-2H-tetrazol-5-yl)amino]-methyl}-5-ethynyl-pyridin-2-yl)cyclopentylmethylethylamine

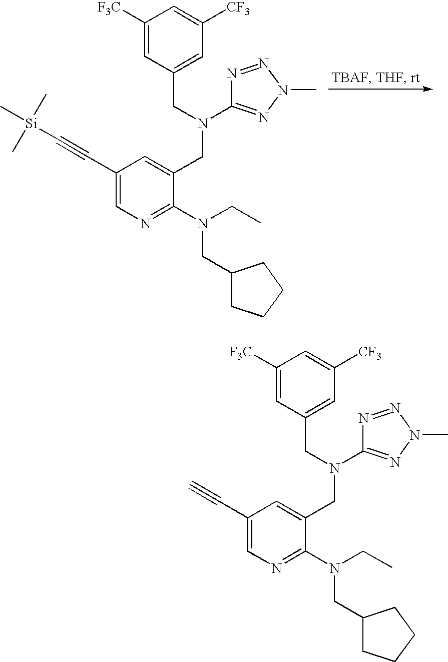

To a solution of (3-{[(3,5-bistrifluoromethylbenzyl)(2-methyl-2H-tetrazol-5-yl)-amino]methyl}-5-trimethylsilanylethynylpyridin-2-yl)cyclopentylmethylethylamine-(96 mg, 0.14 mmol) in THF (1.5 mL), is added tetrabutylammonium fluoride (0.19 mL, 0.19 mmol, 1.0 M in THF). After stirring at room temperature for 5 min, H₂O is added to the reaction mixture. The mixture is extracted with EtOAc. The organic layer is dried and concentrated under reduced pressure. The resulting residue purified by silica gel flash chromatography to give (3-{[(3,5-bistrifluoromethylbenzyl)-(2-methyl-2H-tetrazol-5-yl)amino]-methyl}-5-ethynyl-pyridin-2-yl)cyclopentylmethylethylamine as colorless oil (81 mg, 0.14 mmol; >99%); ESI-MS m/z: 566 [M+1]⁺, Retention time: 2.30 min.

### Example 11

### Synthesis of 5-{[(3,5-bistrifluoromethylbenzyl)(2-methyl-2H-tetrazol-5-yl)amino]methyl}-6-(cyclopentylmethylethylamino)nicotinonitrile

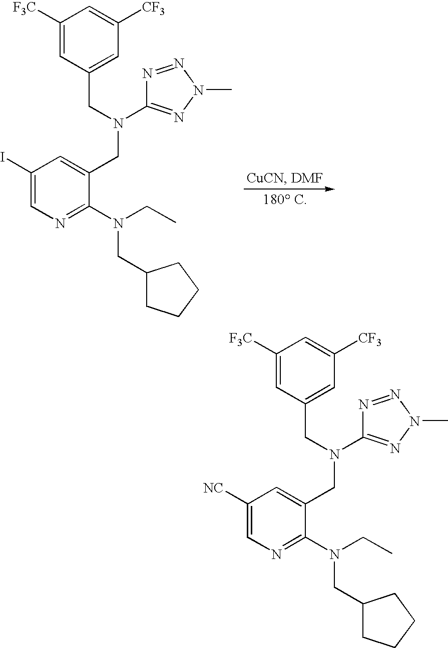

A mixture of (3-{[(3,5-bistrifluoromethylbenzyl)(2-methyl-2H-tetrazol-5-yl)amino]methyl}-5-bromopyridin-2-yl)cyclopentylmethylethylamine (100 mg, 0.16 mmol) and CuCN (42 mg, 0.48 mmol) in DMF (1.0 mL) is heated at 180° C. for 12 h. After cooling down to room temperature, 28% NH₃ solution is added to the reaction mixture. The organic layer is washed with H₂O, dried and concentrated under reduced pressure. The resulting residue is purified by silica gel flash chromatography to give 5-{[(3,5-bistrifluoromethylbenzyl)(2-methyl-2H-tetrazol-5-yl)amino]methyl}-6-(cyclopentylmethylethylamino)nicotinonitrile as colorless oil (62 mg, 0.11 mmol; 73%); ESI-MS m/z: 567 [M+1]⁺, Retention time: 2.43 min.

### Example 12

### Synthesis of 5-{[(3,5-bistrifluoromethylbenzyl)(2-methyl-2H-tetrazol-5-yl)amino]methyl}-6-(cyclopentylmethylethylamino)nicotinic acid

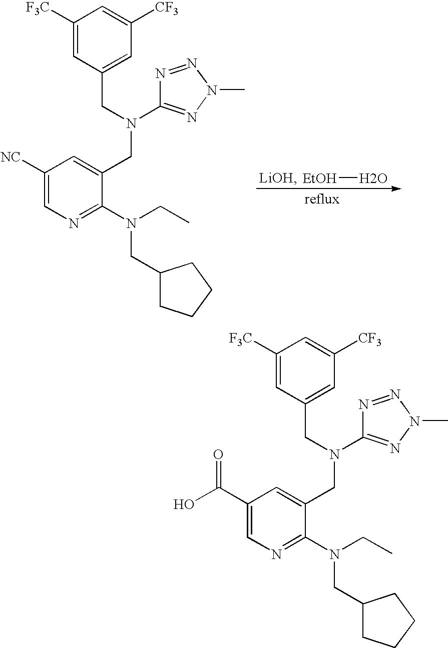

A mixture of give 5-{[(3,5-bistrifluoromethylbenzyl)(2-methyl-2H-tetrazol-5-yl)amino]-methyl}-6-(cyclopentylmethylethylamino)nicotinonitrile (60 mg, 0.10 mmol) and 2M LiOH (1.0 mL, 2.0 mmol) in EtOH (2.0 mL) is heated reflux for 5 h. After cooling down to room temperature, the reaction mixture is acidified by 1M HCl and extracted with EtOAc. The organic layer is washed with H₂O, dried and concentrated under reduced pressure to give 5-{[(3,5-bistrifluoromethylbenzyl)(2-methyl-2H-tetrazol-5-yl)amino]methyl}-6-(cyclopentylmethylethylamino)nicotinic acid as pale yellow oil (58 mg, 0.099, 99%); ESI-MS m/z: 586 [M+1]⁺, Retention time: 2.09 min.

### Example 13

### Synthesis of 5-{[(3,5-bistrifluoromethylbenzyl)(2-methyl-2H-tetrazol-5-yl)amino]methyl}-6-(cyclopentylmethylethylamino)-N-methylnicotinamide

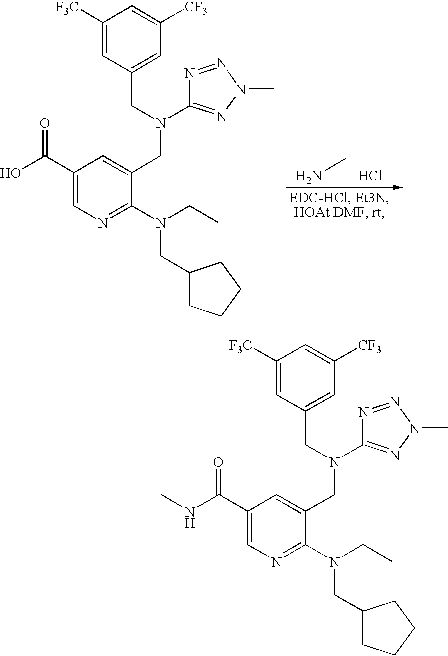

A mixture of give 5-{[(3,5-bistrifluoromethylbenzyl)(2-methyl-2H-tetrazol-5-yl)amino]methyl}-6-(cyclopentylmethylethylamino)nicotinic acid (30 mg, 0.050 mmol), methylamine hydrochloride (10 mg, 0.15 mmol), 1-ethyl-3-(3-dimethylaminopropyl)carbodiimide hydrochloride (17 mg, 0.080 mmol), Et₃N (32 mg, 0.30 mmol) and hydroxy-7-azabenzotriazole (10 mg, 0.050 mmol) in DMF (1.0 mL) is stirred at room temperature overnight. After filtration, the filtrate residue is purified by reverse phase preparative HPLC to give 5-{[(3,5-bistrifluoromethylbenzyl)(2-methyl-2H-tetrazol-5-yl)amino]methyl}-6-(cyclopentylmethylethylamino)-N-methylnicotinamide as pale yellow oil (15 mg, 0.025 mmol; 50%); ESI-MS m/z: 599 [M+1]⁺, Retention time: 1.99 min.

### Example 14

The following compounds are prepared following the procedure of Example 14.

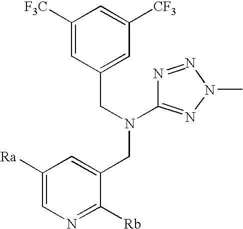

| No. | Ra | Rb | MS | LC (min) |
|---|---|---|---|---|
| 1 | 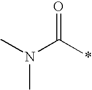 | 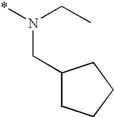 | 613 [M + 1]⁺ | 2.02 |

### Example 15

### Synthesis of (3-{[(3,5-bistrifluoromethyl-benzyl)(2-methyl-2H-tetrazol-5-yl)amino]methyl}-5-nitro-pyridin-2-yl)cyclopentylmethylethylamine

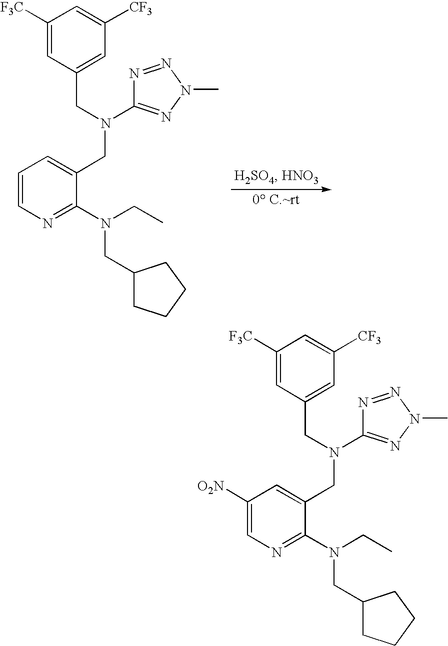

To a solution of (3-{[(3,5-bistrifluoromethylbenzyl)-(2-methyl-2H-tetrazol-5-yl)amino]methyl}pyridin-2-yl)cyclopentylmethylethylamine (100 mg, 0.18 mmol) in H₂SO₄ (0.5 mL), is added fuming HNO₃ (23 mg, 0.36 mmol) at 0° C. After stirring for at the same temperature for 0.5 h, fuming HNO₃ (7 mg, 0.12 mmol) at 0° C. again. The reaction mixture is warmed up to the room temperature and stirred at the same temperature for 1 h. The reaction mixture is pored into ice cold H₂O and turned to be basic condition by 8M NaOH solution. The extracted EtOAc layer is washed with H₂O, dried and concentrated under reduced pressure. The resulting residue is purified by silica gel column chromatography to give (3-{[(3,5-bistrifluoromethyl-benzyl)(2-methyl-2H-tetrazol-5-yl)amino]methyl}-5-nitro-pyridin-2-yl)cyclopentylmethylethylamine as pale yellow oil (73 mg, 0.12 mmol, 69%) ESI-MS m/z: 587 [M+1]⁺, Retention time: 2.45 min.

### Example 16

### Synthesis of 3-{[(3,5-bistrifluoromethylbenzyl)(2-methyl-2H-tetrazol-5-yl)amino]methyl}-N*2*-cyclopentylmethyl-N*2*-ethylpyridine-2,5-diamine

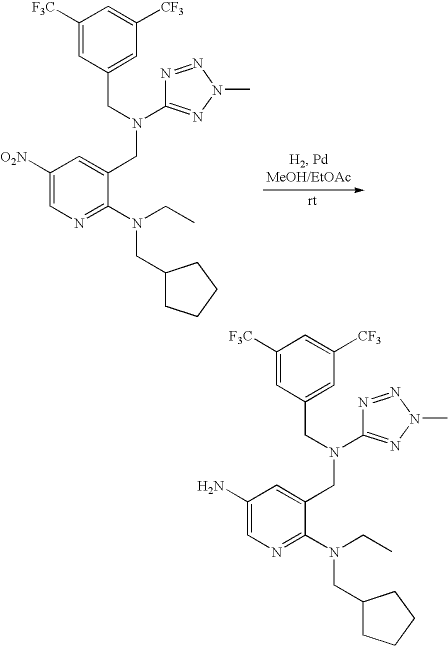

A mixture of (3-{[(3,5-bistrifluoromethyl-benzyl)(2-methyl-2H-tetrazol-5-yl)amino]methyl}-5-nitro-pyridin-2-yl)cyclopentylmethylethylamine (360 mg, 0.61 mmol) with Pd activated carbon ethylenediamine complex (100 mg, 0.033-0.061 mmol, 3.5˜6.5% Pd, Wako pure chemical industries) in MeOH/EtOAc (1:1, 3.0 mL) is stirred under H2 atmosphere at room temperature for 8 h. After filtration, the filtrate residue is purified by reverse phase preparative HPLC to give 3-{[(3,5-bistrifluoromethylbenzyl)(2-methyl-2H-tetrazol-5-yl)amino]methyl}-N*2*-cyclopentylmethyl-N*2*-ethylpyridine-2,5-diamine as pale yellow oil (280 mg, 0.50 mmol; 83%); ESI-MS m/z: 557 [M+1]⁺, Retention time: 1.97 min.

### Example 17

### Synthesis of N-[5-{[(3,5-bistrifluoromethylbenzyl)(2-methyl-2H-tetrazol-5-yl)amino]methyl}-6-(cyclopentylmethylethylamino)pyridin-3-yl]acetamide

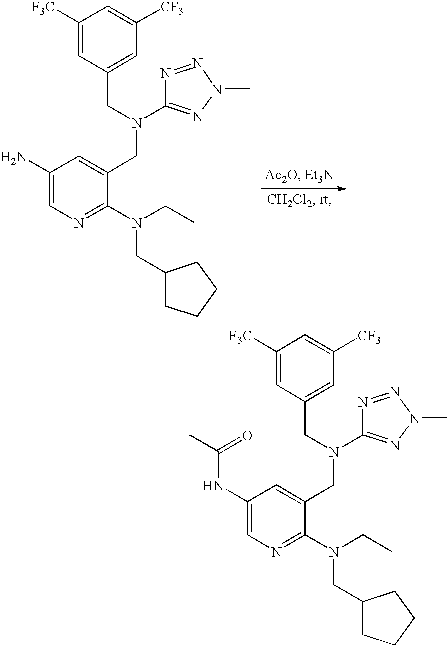

To a solution of 3-{[(3,5-bistrifluoromethylbenzyl)(2-methyl-2H-tetrazol-5-yl)amino]methyl}-N*2*-cyclopentylmethyl-N*2*-ethylpyridine-2,5-diamine (100 mg, 0.18 mmol) and Et₃N (31 mg, 0.29 mmol) in CH₂Cl₂ (1.0 mL), is added acetic anhydride (27 mg, 0.26 mmol) at room temperature. The reaction mixture is stirred at the same temperature for 6 h. After addition of small amount of H₂O, the reaction mixture is concentrated under reduced pressure. The resulting residue is purified by reverse phase preparative HPLC to give N-[5-{[(3,5-bistrifluoromethylbenzyl)(2-methyl-2H-tetrazol-5-yl)amino]methyl}-6-(cyclopentylmethylethylamino)pyridin-3-yl]acetamide as pale yellow oil (23 mg, 0.034 mmol; 21%); ESI-MS m/z: 599 [M+1]⁺, Retention time: 1.94 min.

### Example 18

### Synthesis N-[5-{[(3,5-bis-trifluoromethyl benzyl)-(2-methyl-2H-tetrazol-5-yl)amino]methyl}-6-(cyclopentylmethylethylamino)pyridin-3-yl]acetamide

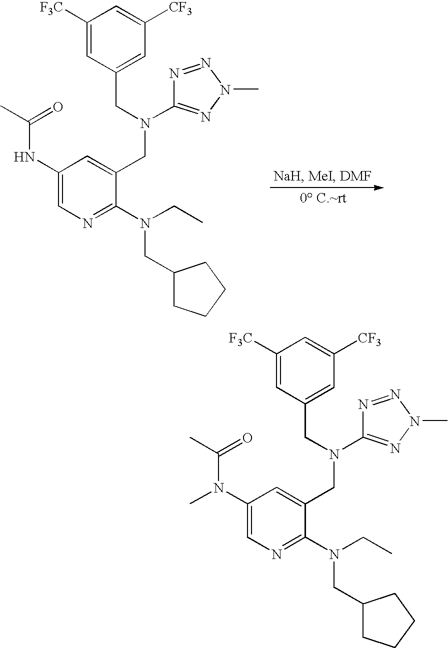

To a solution of N-[5-{[(3,5-bistrifluoromethylbenzyl)(2-methyl-2H-tetrazol-5-yl)amino]methyl}-6-(cyclopentylmethylethylamino)pyridin-3-yl]acetamide (197 mg, 0.32 mmol) in DMF (2.0 mL), is added NaH (60% oil suspension, 17 mg, 0.43 mmol) at 0° C. The reaction mixture is warmed up to room temperature at 0° C. The reaction mixture is stirred at the same temperature for 1 h and quenched by NH₄Cl solution. The resulting residue is purified by reverse phase preparative HPLC to give N-[5-{[(3,5-bis-trifluoromethyl benzyl)-(2-methyl-2H-tetrazol-5-yl)amino]methyl}-6-(cyclopentylmethylethylamino)pyridin-3-yl]acetamide as pale yellow oil (130 mg, 0.21 mmol; 65%); ESI-MS m/z: 613 [M+1]⁺, Retention time: 2.03 min.

### Example 19

### Synthesis of N-[5-{[(3,5-bistrifluoromethylbenzyl)(2-methyl-2H-tetrazol-5-yl)amino]methyl}-6-(cyclopentylmethylethylamino)pyridin-3-yl]methanesulfonamide

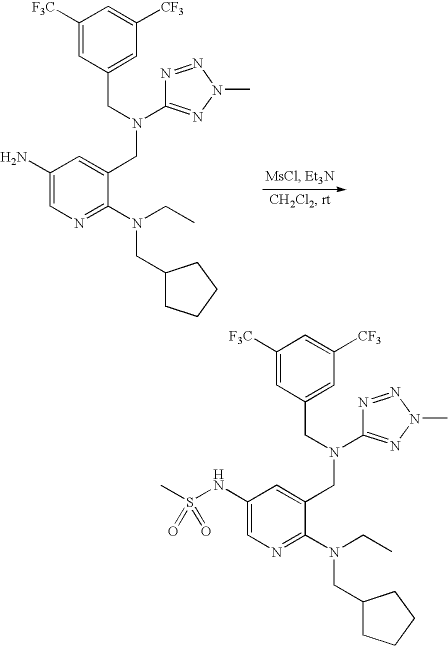

To a solution of 3-{[(3,5-bistrifluoromethylbenzyl)(2-methyl-2H-tetrazol-5-yl)amino]methyl}-N*2*-cyclopentylmethyl-N*2*-ethylpyridine-2,5-diamine (100 mg, 0.18 mmol) and Et₃N (31 mg, 0.29 mmol) in CH₂Cl₂ (1.0 mL), is added methanesulfonyl chloride (30 mg, 0.26 mmol) at room temperature. The reaction mixture is stirred at the same temperature for 6 h. After addition of small amount of H₂O, the reaction mixture is concentrated under reduced pressure. The resulting residue is purified by reverse phase preparative HPLC to give N-[5-{[(3,5-bistrifluoromethylbenzyl)(2-methyl-2H-tetrazol-5-yl)amino]methyl}-6-(cyclopentylmethylethylamino)pyridin-3-yl]methanesulfonamide as pale yellow oil (32 mg, 0.050 mmol; 28%); ESI-MS m/z: 635 [M+1]⁺, Retention time: 2.05 min.

### Example 20

### Synthesis of 5-{[(3,5-bistrifluoromethylbenzyl)(2-methyl-2H-tetrazol-5-yl)amino]methyl}-6-(cyclopentylmethylethylamino)pyridin-3-ol

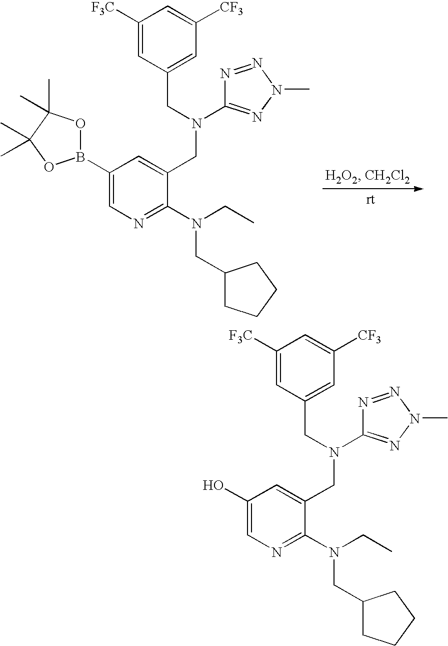

To a solution of 3-{[(3,5-bistrifluoromethylbenzyl)(2-methyl-2H-tetrazol-5-yl)amino]methyl}-5-(4,4,5,5-tetramethyl[1,3,2]dioxaborolan-2-yl)pyridin-2-yl]cyclopentylmethylethylamine (593 mg, 0.88 mmol) in CH₂Cl₂ (5.0 mL), is added H₂O₂ (0.082 mL, 2.6 mmol, 35% solution) at room temperature. After stirring at the same temperature overnight, H₂O is added to the reaction mixture. The CH₂Cl₂ layer is concentrated under reduced pressure. The resulting residue is purified by silica gel flash chromatography to give 5-{[(3,5-bistrifluoromethylbenzyl)(2-methyl-2H-tetrazol-5-yl)amino]methyl}-6-(cyclopentylmethylethylamino)pyridin-3-ol as pale yellow oil (296 mg, 0.47 mmol; 60%); ESI-MS m/z: 558 [M+1]⁺, Retention time: 1.91 min.

### Example 21

### Synthesis of (3-{[(3,5-bistrifluoromethylbenzyl)(2-methyl-2H-tetrazol-5-yl)amino]methyl}-5-ethoxypyridin-2-yl)cyclopentylmethylethylamine

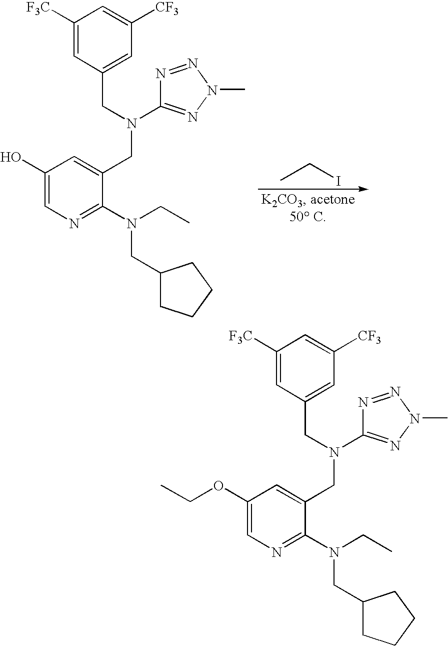

To a solution of 5-{[(3,5-bistrifluoromethylbenzyl)(2-methyl-2H-tetrazol-5-yl)amino]methyl}-6-(cyclopentylmethylethylamino)pyridin-3-ol as pale yellow oil (86 mg, 0.15 mmol) in acetone (2.0 mL), is added idodethane (29 mg, 0.18 mmol) at room temperature. After stirring at 50° C. overnight, the reaction mixture is cooled down to room temperature and treated with H₂O. The mixture is extracted with EtOAc. The organic layer is washed with H₂O, dried and concentrated under reduced pressure. The resulting residue is purified by silica gel flash chromatography to give (3{[(3,5-bistrifluoromethylbenzyl)(2-methyl-2H-tetrazol-5-yl)amino]methyl}-5-ethoxypyridin-2-yl)cyclopentylmethylethylamine as pale yellow oil (53 mg, 0.084 mmol; 45%); ESI-MS m/z: 586 [M+1]⁺, Retention time: 2.05 min.

### Example 22

### Synthesis of (3-{[(3,5-bistrifluoromethylbenzyl)(2-methyl-2H-tetrazol-5-yl)amino]methyl}-5-phenoxy-pyridin-2-yl)cyclopentylmethylethylamine

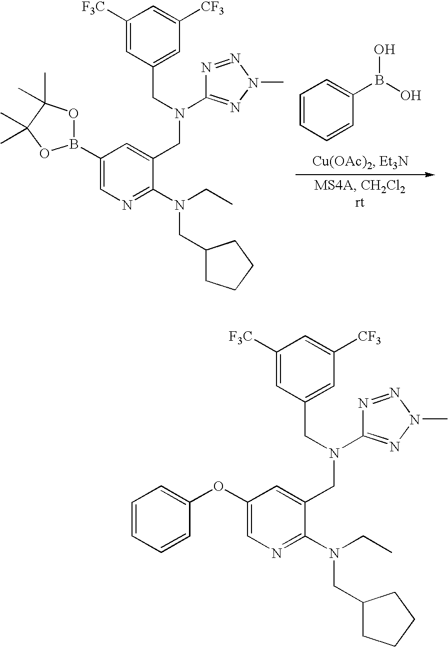

A mixture of 3-{[(3,5-bistrifluoromethylbenzyl)(2-methyl-2H-tetrazol-5-yl)amino]methyl}-5-(4,4,5,5-tetramethyl[1,3,2]dioxaborolan-2-yl)pyridin-2-yl]cyclopentylmethylethylamine (103 mg, 0.18 mmol), phenylboronic acid (34 mg, 0.27 mmol), Cu(OAc)₂ (50 mg, 0.27 mmol), Et₃N (94 mg, 0.92 mol) and molecular sieves 4 Å in CH₂Cl₂ (2.0 mL) is stirred at room temperature overnight. After addition of H₂O and saturated NaHCO₃ solution, the separated organic layer is concentrated under reduced pressure. The resulting residue is purified by silica gel flash chromatography to give (3-{[(3,5-bistrifluoromethylbenzyl)(2-methyl-2H-tetrazol-5-yl)amino]methyl}-5-phenoxy-pyridin-2-yl)cyclopentylmethylethylamine. ESI-MS m/z: 234 [M+1]⁺, Retention time: 2.23 min.

### Example 23

### Synthesis of (3-{[(3,5-bistrifluoromethylbenzyl)(2-methyl-2H-tetrazol-5-yl)amino]methyl}-5-phenoxy-pyridin-2-yl)cyclopentylmethylethylamine

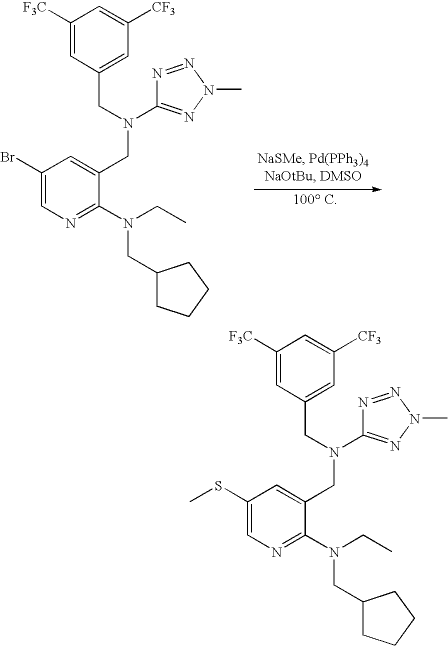

A mixture of (3-{[(3,5-bistrifluoromethylbenzyl)(2-methyl-2H-tetrazol-5-yl)amino]methyl}-5-bromopyridin-2-yl)cyclopentylmethylethylamine (200 mg, 0.16 mmol), sodium thiomethoxide (14 mg, 0.48 mmol), sodium tert-butoxide (92 mg, 0.96 mmol) and Pd(PPh₃)₄ in DMSO (2.0 mL) is heated at 100° C. for 4 h. After cooling down to room temperature, H₂O is added to the reaction mixture. The mixture is extracted with EtOAc. The filtrate is purified by reverse phase preparative HPLC and silica gel flash chromatography to give (3-{[(3,5-bistrifluoromethylbenzyl)(2-methyl-2H-tetrazol-5-yl)amino]methyl}-5-phenoxy-pyridin-2-yl)cyclopentylmethylethylamine as pale yellow oil (3.0 mg, 0.0035 mmol; 2.2%); ESI-MS m/z: 588 [M+1]⁺, Retention time: 2.12 min.

### Example 24

### Synthesis of 3-{[(3,5-bis-trifluoromethyl benzyl)-(2-methyl-2H-tetrazol-5-yl)amino]methyl}-5-methanesulfonylpyridin-2-yl)cyclopentylmethylethylamine

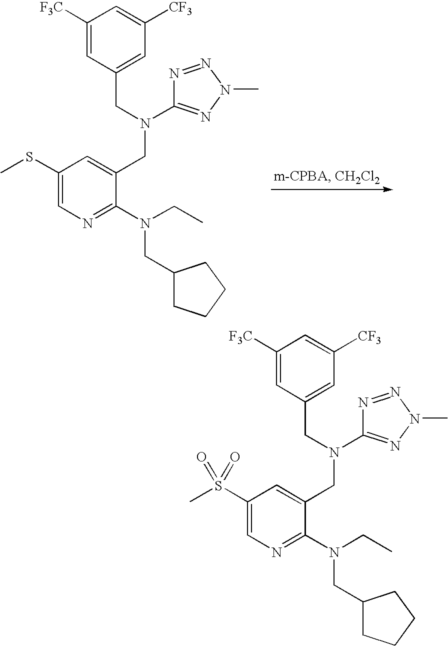

To a solution of the above crude of example 24 (100 mg) in CH₂Cl₂ (1.0 mL), is added m-CPBA (35 mg, 0.20 mmol). After stirring at room temperature for 3 h, Na₂S₂O₃ solution is added to the reaction mixture. The mixture is extracted with CH₂Cl₂. The organic layer is dried and concentrated under reduced pressure. The resulting residue is purified by reverse phase preparative HPLC to give 3-{[(3,5-bis-trifluoromethyl benzyl)-(2-methyl-2H-tetrazol-5-yl)amino]methyl}-5-methanesulfonylpyridin-2-yl)cyclopentylmethylethylamine as pale yellow oil (18 mg, 0.020 mmol); ESI-MS m/z: 620 [M+1]⁺, Retention time: 2.33 min.

### Example 25

### The Following Compounds are Prepared Following the Procedure of Example 21-25

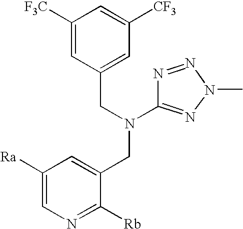

| No. | Ra | Rb | MS | LC (min) |
|---|---|---|---|---|
| 1 | 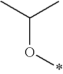 | 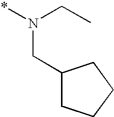 | 600 [M + 1]⁺ | 2.08 |

### Example 26

### Synthesis of (3-{[(3,5-bistrifluoromethylbenzyl)(2-methyl-2H-tetrazol-5-yl)amino]methyl}-5-ethylpyridin-2-yl)cyclopentylmethylethylamine

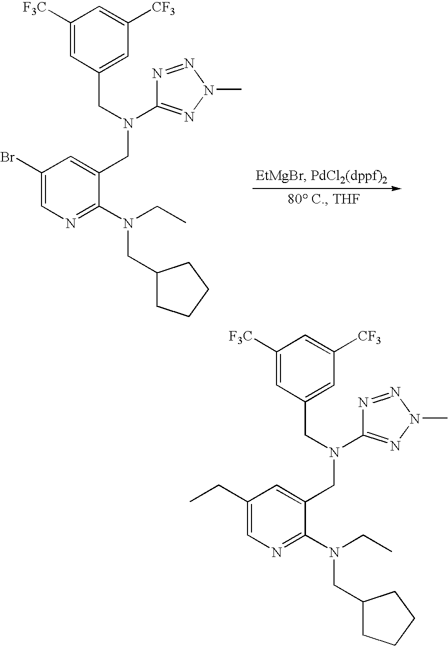

To a solution of (3-{[(3,5-bistrifluoromethylbenzyl)(2-methyl-2H-tetrazol-5-yl)amino]methyl}-5-bromopyridin-2-yl)cyclopentylmethylethylamine (152 mg, 0.25 mmol) and PdCl₂(dppf)₂ (20 mg, 0.025 mmol) in THF (2.0 mL), is added ethyl magnesium bromide (0.86 mL, 0.74 mmol, 0.86M in THF) at 0° C. After stirred at 80° C. for 3 h, H₂O is added to the reaction mixture. The mixture is extracted with EtOAc. The organic layer is washed with H₂O, dried and concentrated under reduced pressure. The resulting residue is purified by silica gel flash chromatography to give (3-{[(3,5-bistrifluoromethylbenzyl)(2-methyl-2H-tetrazol-5-yl)amino]methyl}-5-ethylpyridin-2-yl)cyclopentylmethylethylamine as pale yellow oil (42 mg, 0.074 mmol; 30%); ESI-MS m/z: 570 [M+1]⁺, Retention time: 2.01 min.

### Example 27

### The Following Compounds are Prepared Following the Procedure of Example 26

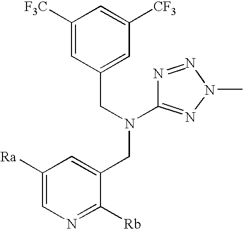

| No. | Ra | Rb | MS | LC (min) |
|---|---|---|---|---|
| 1 | 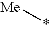 | 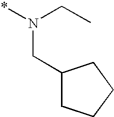 | 556 [M + 1]⁺ | 1.97 |
| 2 | 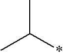 | 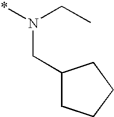 | 584 [M + 1]⁺ | 2.03 |
| 3 | 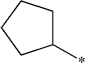 | 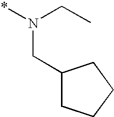 | 610 [M + 1]⁺ | 2.09 |

General HPLC Condition

Column: Waters ACQUITY HPLC BEH C18, 1.7 μM

Mobile phase: CH₃CN/H₂O (0.1% TFA)

### Example 29

### Preparation of the Starting Materials can be Done as Follows

### 1). Synthesis of 2-(cyclopentylmethylethylamino)pyridine-3-carbaldehyde

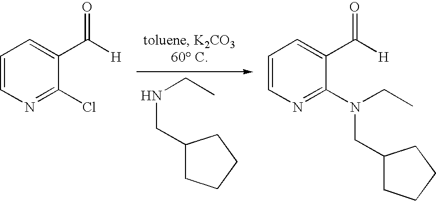

A mixture of cyclopentylmethylethylamine (2.7 g, 19 mmol, CAS: 4492-37-9) and 2-chloro-pyridine-3-carbaldehyde (3.0 g, 24 mmol, CAS: 36404-88-3) and K₂CO₃ (3.3 g, 24 mmol) in toluene is heated at 130° C. for 24 hours. After filtration and concentration under reduced pressure, the filtrate is purified by silica gel column chromatography to give 2-(cyclopentylmethylethylamino)pyridine-3-carbaldehyde as pale yellow oil (3.5 g, 15 mmol, 79% yield).

ESI-MS m/z: 233 [M+1]⁺, Retention time: 1.64 min.

### 2). Synthesis of (3-{[(3,5-bistrifluoromethylbenzyl)(2-methyl-2H-tetrazol-5-yl)amino]methyl}pyridin-2-yl)cyclopentylmethylethylamine

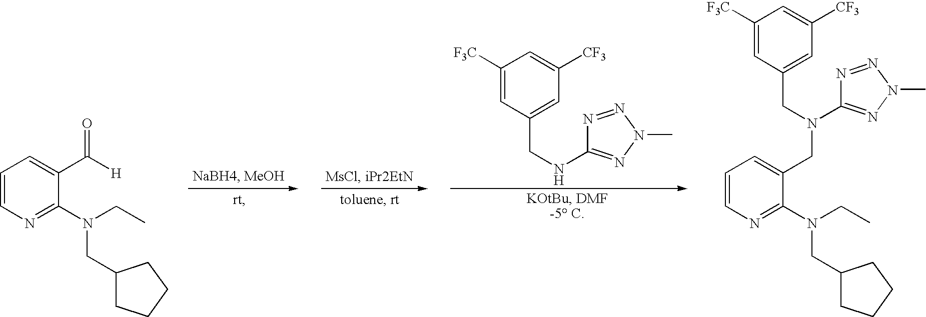

To a solution of 2-(cyclopentylmethylethylamino)pyridine-3-carbaldehyde (500 mg, 2.1 mmol) in MeOH, is added NaBH₄ (80 mg, 2.1 mmol) at room temperature. After stirring at the same temperature for 1 h, the reaction mixture is quenched by NH₄Cl solution and extracted with CH₂Cl₂ and concentrated with toluene under reduced pressure. A mixture of the resulting residue, methanesulfonylchloride (285 mg, 2.5 mmol) and diisopropyl-ethylamine (361 mg, 2.8 mmol) in toluene is stirred at room temperature for 1 h. After ice-cold NaHCO₃ solution is added, the mixture is extracted with EtOAc. The extracted organic layer is washed, dried, filtrated and concentrated under reduced pressure. The resulting residue is added to a solution of [3,5-bis(trifluoromethyl)benzyl](2-methyl-2H-tetrazol-5-yl)amine (683 mg, 2.1 mmol) and potassium tert-butoxide (256 mg, 2.1 mmol) in DMF (2.0 mL), which is prepared in advance at −5° C. for 0.5 h. After stirring at the same temperature for 2.5 h, the reaction mixture is quenched with ice cold water and extracted with EtOAc. The extracted organic layer is washed, dried, filtrated and concentrated under reduced pressure. The resulting residue is purified by silica gel column chromatography to give (3-{[(3,5-bistrifluoromethylbenzyl)(2-methyl-2H-tetrazol-5-yl)amino]methyl}pyridin-2-yl)cyclopentylmethylethylamine as pale yellow oil (830 mg, 1.5 mmol, 71% yield). ESI-MS m/z: 542 [M+1]⁺, Retention time: 1.93 min.

### 3). Synthesis of (3-{[(3,5-bistrifluoromethylbenzyl)(2-methyl-2H-tetrazol-5-yl)amino]methyl}-5-bromo-pyridin-2-yl)cyclopentylmethylethylamine

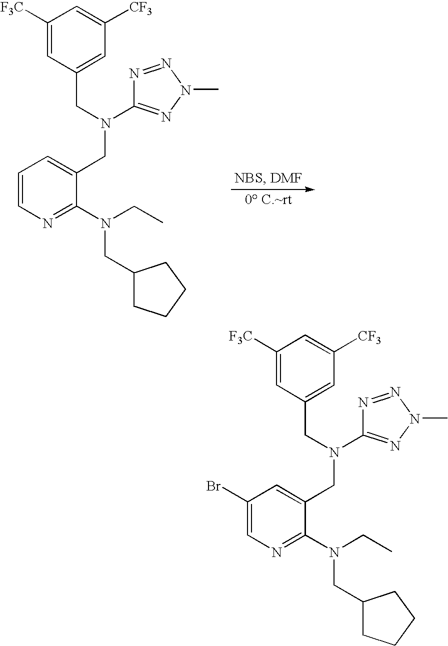

To a solution of (3-{[(3,5-bistrifluoromethylbenzyl)(2-methyl-2H-tetrazol-5-yl)amino]methyl}pyridin-2-yl)cyclopentylmethylethylamine (100 mg, 0.18 mmol) in DMF (0.5 mL), is added N-bromosuccinimide (38 mg, 0.21 mmol) at 0° C. After stirring at the same temperature for 1 h, the reaction mixture is warmed up to the room temperature and diluted with EtOAc. The extracted organic layer is washed, dried, filtrated and concentrated under reduced pressure. The resulting residue is purified by silica gel column chromatography to give (3-{[(3,5-bistrifluoromethylbenzyl)(2-methyl-2H-tetrazol-5-yl)amino]methyl}-5-bromopyridin-2-yl)cyclopentylmethylethylamine as pale yellow oil (90 mg, 0.14 mmol, 81%) ESI-MS m/z: 619 [M+1]⁺, Retention time: 2.47 min.

### 4). Synthesis of 3-{[(3,5-bistrifluoromethylbenzyl)(2-methyl-2H-tetrazol-5-yl)amino]methyl}-5-(4,4,5,5-tetramethyl[1,3,2]dioxaborolan-2-yl)pyridin-2-yl]cyclopentylmethylethylamine

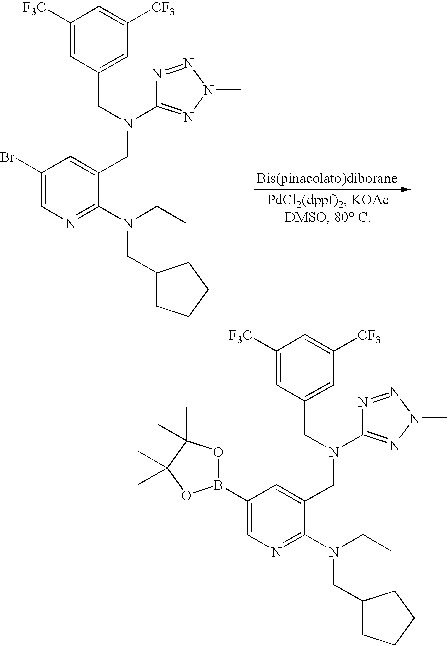

A mixture of (3-{[(3,5-bistrifluoromethylbenzyl)(2-methyl-2H-tetrazol-5-yl)amino]methyl}-5-bromopyridin-2-yl)cyclopentylmethylethylamine (716 mg, 1.15 mmol), KOAc (340 mg, 3.46 mmol), bis(pinacolato)diborone (440 mg, 1.73 mmol) and PdCl₂(dppf)₂ (94 mg, 0.11 mmol) in DMSO (5.0 mL) is heated at 80° C. for 3 h. After cooling down to room temperature, H₂O is added to the reaction mixture. The mixture is extracted with EtOAc. The organic layer is washed with H₂O, dried, filtrated and concentrated under reduced pressure. The resulting residue is purified by silica gel flash chromatography to give 3-{[(3,5-bistrifluoromethylbenzyl)(2-methyl-2H-tetrazol-5-yl)amino]methyl}-5-(4,4,5,5-tetramethyl[1,3,2]dioxaborolan-2-yl)pyridin-2-yl]cyclopentylmethylethylamine as pale yellow oil (756 mg, 0.88 mmol; 98%); ESI-MS m/z: 586 [M−82]⁺, Retention time: 1.87 min.

1. A compound of formula (I): 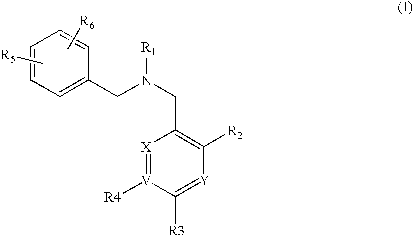
X and Y are independently CH or N;
V is C or N, provided that when V is N, R₄ is hydrogen;
R₁ is heteroaryl, heterocyclyl, aryl, alkoxycarbonyl, alkanoyl, or alkyl, each is optionally substituted with one to three substituents selected from alkyl, hydroxy, halogen, nitro, carboxy, thiol, cyano, HSO₃—, cycloalkyl, alkenyl, alkoxy, cycloalkoxy, alkenyloxy, alkoxycarbonyl, carbamimidoyl, alkyl-S—, alkyl-SO—, alkyl-SO₂—, amino, H₂N—SO₂—, alkanoyl, heterocyclyl;
R₂ is hydrogen, alkyl, halogen, cycloalkyl, cycloalkyl-alkyl-, aryl alkoxy, or (R₇)(R₈)N—;
wherein R₇ and R₈ are independently alkyl, cycloalkyl, alkanoyl, cycloalkyl-C(O)—, or R₈-alkyl-, each of which is optionally substituted by one to three substituents selected from alkyl, alkanoyl, hydroxy, alkoxy, or halogen;
wherein R₅ is aryl, cycloalkyl, heterocyclyl, R₁₀—C(O)—;
wherein R₁₀ is hydrogen, hydroxy, alkyl, heterocyclyl, (Rₐ)(R_{b})N— or cycloalkyl;
wherein Rₐ and R_{b} is alkyl, cycloalkyl, alkanoyl, cycloalkyl-C(O)—,
R₇ and R₈ takers together with the nitrogen to which they are attached optionally form a 3 to 8 membered ring; or
R₂ is heterocyclyl that is optionally substituted by one to three substituents selected from alkyl, hydroxy, aryl, aryl-alkyl-, cycloalkyl, heteroaryl, heterocyclyl, halogen, carboxy, amide, SO₂NH—, alkyl-SO₂—NH—, alkyl-NH—SO₂—, or R₁₀—C(O)—, wherein R₁₀ is hydrogen, hydroxy, alkyl, heterocyclyl, (R₇)(R₈)N— or cycloalkyl;
R₃ is aryl or heteroaryl, each is optionally substituted by one to two substituents selected from halogen, alkyl, alkoxy, or alkyl-SO₂—;
R₄ is substituted aryl or heteroaryl, each is substituted by one to two substituents selected from halogen, alkyl, alkoxy, or alkyl-SO₂—; or
R₃ and R₄ are independent hydrogen, alkyl, alkoxy, halogen, heterocyclyl, alkyl-S—, alkyl-SO₂—, aryloxy, cyano, nitro, HO—C(O)—, or hydroxy; or
R₃ and R₄ are independently (R₁₁)(R₁₂)N—C(O)—, (R₁₃)(R₁₄)N—, wherein R₁₁ and R₁₂ are independently hydrogen, alkyl, aryl, heteroary, or aryl-alkyl-; R₁₃ and R₁₄ are independently hydrogen, alkyl, alkyl-C(O)—, or alkyl-SO₂—;
R₁₃ and R₁₄ taken together with the nitrogen to which they are attached optionally form a 3 to 8 membered ring;
R₅ and R₆ are independently hydrogen, alkyl, haloalkyl, halogen, cyano, nitro, hydroxy, or alkoxy; or
R₆ is aryl or heteroaryl; or
a pharmaceutically acceptable salt thereof; or an optical isomer thereof; or a mixture of optical isomers.

2. The compound of claim 1, wherein X and Y are independently CH or N; V is C or N, provided that when V is N, R4 is hydrogen;
R₁ is (5-9)-membered heteroaryl, (5-9)-membered heterocyclyl, (C₆-C₁₀) aryl, or (C₁-C₇) alkyl, each is optionally substituted with one substituent selected from (C₁-C₇) alkyl, hydroxy, halogen, nitro, carboxy, thiol, cyano, HSO₃—, (C₃-C₇) cycloalkyl, (C₁-C₇) alkenyl, (C₁-C₇) alkoxy, (C₃-C₇) cycloalkoxy, (C₁-C₇) alkenyloxy, (C₁-C₇) alkoxycarbonyl, carbamimidoyl, (C₁-C₇) alkyl-S—, (C₁-C₇) alkyl-SO—, (C₁-C₇) alkyl-SO₂—, amino, H₂N—SO₂—, (C₁-C₇) alkanoyl, (5-9)-membered heterocyclyl;
R₂ is hydrogen, (C₁-C₇) alkyl, halogen, (C₃-C₇) cycloalkyl, (C₁-C₇) cycloalkyl-(C₁-C₇) alkyl, (C₆-C₁₀) aryl, (C₁-C₇) alkoxy, (5-9)-membered heterocyclyl, or (R₇)(R₈)N—, wherein R₇ and R₈ are independently (C₁-C₇) alkyl, hydroxy, halogen, (C₁-C₇) alkyl-C(O)—, (C₃-C₇) cycloalkyl-C(O)—, or R₉—(C₁-C₇) alkyl-, wherein R₉ is (C₃-C₇) cycloalkyl, (C₅-C₁₀) aryl, (5-9)-membered heterocyclyl, or R₁₀—C(O)—, wherein R₁₀ is (C₃-C₇) cycloalkyl, (C₁-C₇) alkyl, (5-9)-membered heterocyclyl (Rₐ)(R_{b})N—, hydroxy, or hydrogen;
wherein Rₐ and R_{b} is (C₁-C₇) alkyl, (C₃-C₇) cycloalkyl, (C₁-C₇) alkanoyl, (C₃-C₇) cycloalkyl-C(O)—,
R₇ and R₈ taken together with the nitrogen to which they are attached optionally form a 3 to 8 membered ring; or
R₂ is (5-9)-membered heterocyclyl that is optionally substituted by one to two substituents selected from (C₁-C₇) alkyl, hydroxy, (C₆-C₁₀) aryl, (C₆-C₁₀) aryl-(C₁-C₇) alkyl-, (C₃-C₇) cycloalkyl, (5-9)-membered heteroaryl carboxy, amide, SO₂—NH—, (C₁-C₇) alkyl-SO₂—NH—, (C₁-C₇) alkyl-NH—SO₂—, halogen, or R₁₀—C(O)—, wherein R₁₀ is (C₁-C₇) cycloalkyl, (C₁-C₇) alkyl, hydroxy, or hydrogen;
R₃ is (C₆-C₁₀) aryl or (5-9)-membered heteroaryl, each is optionally substituted by one to two substituents selected from halogen, (C₁-C₇) alkyl, (C₁-C₇) alkoxy, or (C₁-C₇) alkyl-SO₂—;
R₄ is substituted (C₆-C₁₀) aryl or (5-9)-membered heteroaryl, each is optionally substituted by one to two substituents selected from halogen, (C₁-C₇) alkyl, (C₁-C₇) alkoxy, or (C₁-C₇) alkyl-SO₂—; or
R₃ and R₄ are independently hydrogen, (C₁-C₇) alkyl, (C₁-C₇) alkoxy, halogen, (5-9)-membered heterocyclyl, (C₁-C₇) alkyl-S—, (C₁-C₇) alkyl-SO₂—, (C₆-C₁₀) aryloxy, cyano, nitro, HO—C(O)—, or hydroxy; or
R₃ and R₄ are independently (R₁₀)(R₁₁)N—C(O)—, (R₁₂)(R₁₃)N—, wherein R₁₀ and R₁₁ are independently hydrogen or (C₁-C₇) alkyl, (C₆-C₁₀) aryl, (C₆-C₁₀) aryl-(C₁-C₇) alkyl-; R₁₂ and R₁₃ are independently hydrogen, (C₁-C₇) alkyl, (C₁-C₇) alkyl-C(O)—, or (C₁-C₇) alkyl-SO₂—;
R₁₂ and R₁₃ taken together with the nitrogen to which they are attached optionally form a 3 to 8 membered ring;
R₅ and R₆ are independently hydrogen, (C₁-C₇) alkyl, (C₁-C₇) haloalkyl, halogen, cyano, nitro, hydroxy, or (C₁-C₇) alkoxy; or
R₆ is (C₆-C₁₀) aryl or (5-9)-membered heteroaryl; or
a pharmaceutically acceptable salt thereof; or an optical isomer thereof; or a mixture of optical isomers.

3. A method of inhibiting CETP activity in a subject, comprising:
administering to the subject a therapeutically effective amount of the compound of formula (I) according to claim 1.

4. A method of treating a disorder or a disease in a subject mediated by CETP or responsive to inhibition of CETP, comprising:
administering to the subject a therapeutically effective amount of the compound of formula (I) according to claim 1.

5. The method of claim 4, wherein the disorder or the disease is selected from hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity or endotoxemia etc.

8. A pharmaceutical composition comprising:
a therapeutically effective amount of a the compound of formula (I) according to claim 1 and
one or more pharmaceutically acceptable carriers.

7. A pharmaceutical composition, comprising:
a therapeutically effective amount of the compound according to claim 1 and
one or more therapeutically active agents selected from the group consisting of a:
(i) HMG-Co-A reductase inhibitor or a pharmaceutically acceptable salt thereof,
(ii) angiotensin II receptor antagonist or a pharmaceutically acceptable salt thereof,
(iii) angiotensin converting enzyme (ACE) Inhibitor or a pharmaceutically acceptable salt thereof,
(iv) calcium channel blocker or a pharmaceutically acceptable salt thereof.
(v) aldosterone synthase inhibitor or a pharmaceutically acceptable salt thereof,
(vi) aldosterone antagonist or a pharmaceutically acceptable salt thereof,
(vii) dual angiotensin converting enzyme/neutral endopeptidase (ACE/NEP) inhibitor or a pharmaceutically acceptable salt thereof,
(viii) endothelin antagonist or a pharmaceutically acceptable salt thereof,
(ix) renin Inhibitor or a pharmaceutically acceptable salt thereof,
(x) diuretic or a pharmaceutically acceptable salt thereof,
(xi) an ApoA-I mimic, and
(Xii) a DGAT Inhibitor.

8-10. (canceled)
